(19) United States
(12) Patent Application Publication
(10) Pub. No.: US 2011/0124569 A1
(43) Pub. Date: May 26, 2011

(54) MACROCYCLIC COMPOUNDS, PROTEASE INHIBITION, AND METHODS OF TREATMENT

(75) Inventors: Hendrik Luesch, Gainesville, FL (US); Kanchan Taori, Gainesville, FL (US); Valerie J. Paul, Fort Pierce, FL (US); Susan Matthew, Gainesville, FL (US)

(73) Assignee: UNIVERSITY OF FLORIDA RESEARCH FOUNDATION, INC., Gainesville, FL (US)

(21) Appl. No.: 12/677,093

(22) PCT Filed: Sep. 9, 2008
(86) PCT No.: PCT/US2008/010560

§ 371 Date: Jan. 31, 2011

Related U.S. Application Data
(60) Provisional application No. 60/970,990, filed on Sep. 9, 2007.
(60) Provisional application No. 61/135,941, filed on Jul. 25, 2008.

(51) Int. Cl. A61K 38/12; C07K 7/06; A61P 11/00; A61P 19/02; A61P 9/10; A61P 17/00

(52) U.S. Cl. 514/16.6; 530/328; 530/329; 514/21.1; 514/18.6

(57) ABSTRACT

The instant invention describes macrocyclic depsipeptide lyngbyastatins, and methods of treating disorders such as COPD, emphysema, rheumatoid arthritis, and aging related disorders.

### CROSS-REFERENCE TO RELATED APPLICATIONS

This application claims the benefit of U.S. Provisional Patent Application Nos. 60/970,990, filed Sep. 9, 2007, and 61/135,941, filed Jul. 25, 2008, the entire teachings of which are hereby incorporated by reference.

### STATEMENT OF RIGHTS TO INVENTIONS MADE UNDER FEDERALLY SPONSORED RESEARCH

This work was supported in part by a NOAA, Office of Sea Grant, U.S Department of Commerce Grant No. NA06OAR4170014. The government has certain rights in the invention.

### BACKGROUND

Marine cyanobacteria are a rich source of structurally intriguing bioactive compounds (Gerwick, W. H.; Tan, L. T.; Sitachitta, N. Alkaloids Chem. Biol. 2001, 57, 75-184) and also appear to be the true source of many sea hare isolates, including dolastatins (Luesch, H.; Harrigan, G. G.; Goetz, G.; Horgen, F. D. Curr. Med. Chem. 2002, 9, 1791-1806). Recently reported has been the isolation of a new analogue of dolastatin 13 (Pettit, G. R.; Kamano, Y.; Herald, C. L.; Dufresne, C.; Cerny, R. L.; Herald, D. L.; Schmidt, J. M.; Kizu, H. J. Am. Chem. Soc. 1989, 111, 5015-5017) with protease inhibitory activity, lyngbyastatin 4, from the marine cyanobacterium Lyngbya confervoides collected off the South Florida Atlantic coast (Matthew, S.; Ross, C.; Rocca, J. R; Paul, V. J; Luesch, H. J. Nat. Prod. 2007, 70, 124-127). Additionally, certain marine cyanobacteria produce a wide array of secondary metabolites including peptides and depsipeptides. Cyanobacterial metabolites commonly contain modified or unusual amino acid units, which presumably confer resistance to proteolytic degradation and thus contribute to bioactivity. Concomitantly, such structural features may allow them to interact with proteases (Lee, A. Y. et al. J. Chem. Biol. 1994, 1, 113; Sandler B. et al. J. Am. Chem. Soc. 1998, 120, 595).

Elastase overactivity is involved in tissue destruction and inflammation characteristic of various diseases, such as chronic obstructive pulmonary disease, hereditary emphysema, cystic fibrosis, adult respiratory distress syndrome, and ischemic-reperfusion injury (Tremblay, G. M.; Janelle, M. F.; Bourbonnais, Y. Curr. Opin. Investig. Drugs 2003, 4, 556-565). It is also believed to contribute to cutaneous wrinkling (Tsuji, N.; Moriwaki, S.; Suzuki, Y.; Takema, Y.; Imokawa, G. Photochem. Photobiol., 2001, 74, 283-290). Consequently, enzyme inhibition has been recognized as a valid therapeutic approach for various indications, and drug discovery efforts have resulted in several small molecules that have entered clinical trials (Ohbayashi, H. Exp. Opin. Ther. Patents 2005, 15, 759-771).

### BRIEF SUMMARY OF THE INVENTION

The invention is directed towards macrocyclic compounds, including depsipeptide lyngbyastatins and kempopeptins, methods of inhibiting elastase using lyngbyastatins, and methods of treating disorders.

In one embodiment, the invention provides a compound according to Formula I:

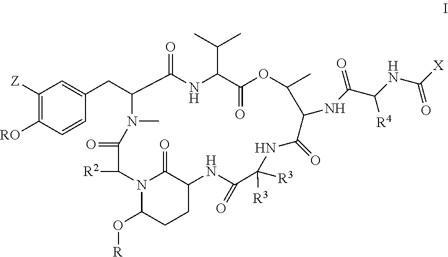

wherein:

each R is independently H or optionally substituted alkyl;

X₁ is optionally substituted aryl, optionally substituted heteroaryl, optionally substituted cycloalkyl, optionally substituted heterocycloalkyl, —OR^{a}, —NR^{a}R^{a}, —C(O)R^{a}, or —OC(O)R^{a};

R^{a}, for each instance is independently selected from H, an optionally substituted alkyl, an optionally substituted cycloalkyl, an optionally substituted heterocycloalkyl, an optionally substituted aryl, an optionally substituted heteroaryl, haloalkyl, hydroxylalkyl, amino, or mono- or di-substituted amine;

X is alkyl, N-acetylpyrrolidin-2-yl, or

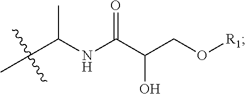

R¹ is selected from H, —S(O)_{q}R^{b}, optionally substituted alkyl, optionally substituted carbocyclic aryl, optionally substituted heteroaryl, optionally substituted cycloalkyl, or optionally substituted heterocyclic;

R² is alkyl, optionally substituted with aryl;

each R³ is independently H, alkyl optionally substituted with NH₂, or both R³ taken together with the carbon to which they are attached form C═CHR;

each R⁴ is independently alkyl optionally substituted with X₁;

R^{b} is H, Na, or K;

q is an integer from 0, 1, 2 or 3;

each Z is independently H or halogen;

and pharmaceutically acceptable salts, solvate, or hydrate thereof.

In one embodiment, the invention provides a compound according to Formula Ia:

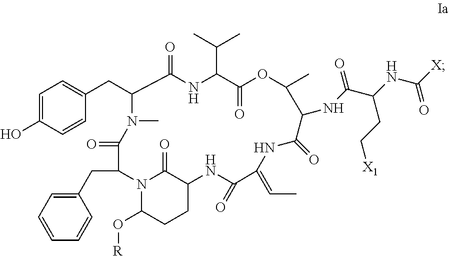

wherein:

R is H or optionally substituted alkyl;

X₁ is optionally substituted aryl, optionally substituted heteroaryl, optionally substituted cycloalkyl, optionally substituted heterocycloalkyl, —OR^{a}, —NR^{a}R^{a}, —C(O)R^{a}, or —OC(O)R^{a};

R^{a}, for each instance is independently selected from H, an optionally substituted alkyl, an optionally substituted cycloalkyl, an optionally substituted heterocycloalkyl, an optionally substituted aryl, an optionally substituted heteroaryl, haloalkyl, hydroxylalkyl, amino, or mono- or di-substituted amine;

X is alkyl or

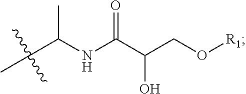

R¹ is selected from H, —S(O)_{q}R^{b}, optionally substituted alkyl, optionally substituted carbocyclic aryl, optionally substituted heteroaryl, optionally substituted cycloalkyl, or optionally substituted heterocyclic;

R^{b} is H, Na, or K;

q is an integer from 0, 1, 2 or 3;

and pharmaceutically acceptable salts, solvate, or hydrate thereof.

In certain instances, the compounds of the invention are selected from the following:

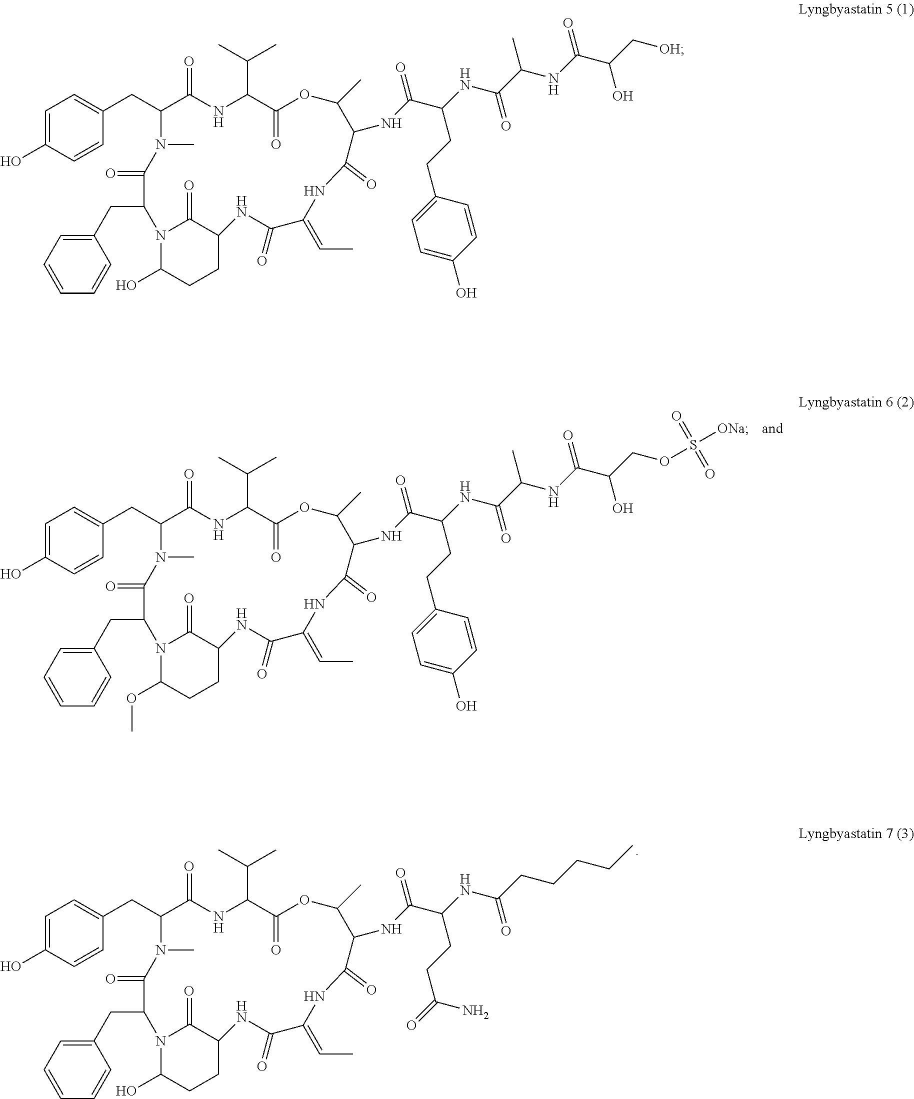

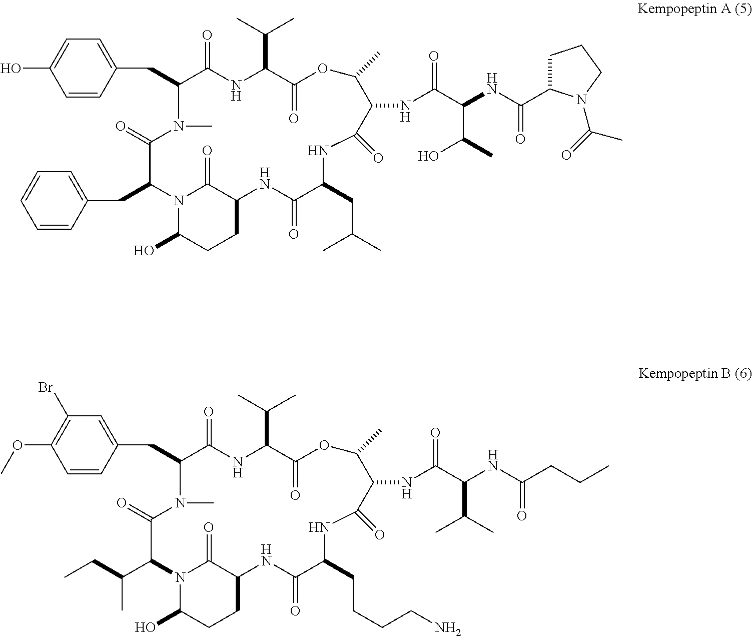

In another aspect, the invention provides a pharmaceutical composition comprising the compound of formula I (e.g., formula I, Ia, etc.) and a pharmaceutically acceptable carrier.

In other aspects, the invention provides a method of modulating the activity of a protease in a subject, comprising contacting the subject with a compound of formula I, in an amount and under conditions sufficient to modulate protease activity.

In another aspect, the invention provides a method of modulating the activity or overactivity of elastase in a subject, comprising contacting the subject with a compound of formula I, in an amount and under conditions sufficient to modulate elastase activity.

In one aspect, the invention provides a method of treating a subject suffering from or susceptible to an elastase overactivity related disorder or disease, comprising administering to the subject an effective amount of a compound or pharmaceutical composition of formula I.

In another aspect, the invention provides a method of treating a subject suffering from or susceptible to an elastase overactivity related disorder or disease, wherein the subject has been identified as in need of treatment for an elastase overactivity related disorder or disease, comprising administering to said subject in need thereof, an effective amount of a compound or pharmaceutical composition of formula I, such that said subject is treated for said disorder.

In a specific aspect, the invention provides a method of treating chronic obstructive pulmonary disease (COPD), lung tissue injury, emphysema, hereditary emphysema, rheumatoid arthritis, cystic fibrosis, adult respiratory distress syndrome, reperfusion injury, ischemic-reperfusion injury, or an aging-related skin disorder, comprising administering to said subject in need thereof, an effective amount of Lyngbyastatin 5, Lyngbyastatin 6, Lyngbyastatin 7, Kempopeptin A, Kempopeptin B, or pharmaceutically acceptable salts thereof.

In a specific aspect, the invention provides a method of treating trypsin activity (or disease, disorder or symptom thereof associated with trypsin activity) comprising administering to said subject in need thereof, an effective amount of Kempopeptin B or pharmaceutically acceptable salts thereof.

### BRIEF DESCRIPTION OF THE DRAWINGS

FIG. 1 shows an analysis of Lyngbyastatin 5 (1) and Lyngbyastatin 6 (2) by ¹H NMR, COSY, TOCSY, ROESY, HSQC, and HMBC spectra recorded in DMSO-d₆ which revealed the presence of alanine, valine, threonine, phenylalanine, N-methyltyrosine, glyceric acid (Ga), homotyrosine (Htyr), 2-amino-2-butenoic acid (Abu) and 3-amino-6-hydroxy-2-piperidone (Ahp).

FIG. 2 shows an analysis of compound 3 by ¹H NMR, ¹³C NMR, HMQC, COSY, TOCSY, and HMBC spectra, which revealed the presence of valine, threonine, phenylalanine, N-methyltyrosine, glutamine, hexanoic acid (Ha), Abu and Ahp moieties. Analysis of ¹H NMR, ¹³C NMR, HMQC, COSY, TOCSY, and HMBC spectra also revealed the presence of valine, threonine, phenylalanine, N-methyltyrosine, glutamine, hexanoic acid (Ha), Abu and Ahp moieties.

### DETAILED DESCRIPTION

### Definitions

In order that the invention may be more readily understood, certain terms are first defined here for convenience.

As used herein, the term “treating” a disorder encompasses preventing, ameliorating, mitigating and/or managing the disorder and/or conditions that may cause the disorder. The terms “treating” and “treatment” refer to a method of alleviating or abating a disease and/or its attendant symptoms. In accordance with the present invention “treating” includes preventing, blocking, inhibiting, attenuating, protecting against, modulating, reversing the effects of and reducing the occurrence of e.g., the harmful effects of a disorder.

As used herein, “inhibiting” encompasses preventing, reducing and halting progression.

The term “modulate” refers to increases or decreases in the activity of a cell in response to exposure to a compound of the invention.

The terms “isolated,” “purified,” or “biologically pure” refer to material that is substantially or essentially free from components that normally accompany it as found in its native state. Purity and homogeneity are typically determined using analytical chemistry techniques such as polyacrylamide gel electrophoresis or high performance liquid chromatography. A protein that is the predominant species present in a preparation is substantially purified. Particularly, it means that the nucleic acid or protein is at least 85% pure, more preferably at least 95% pure, and most preferably at least 99% pure.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

A “peptide” is a sequence of at least two amino acids. Peptides can consist of short as well as long amino acid sequences, including proteins.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

The term “protein” refers to series of amino acid residues connected one to the other by peptide bonds between the alpha-amino and carboxy groups of adjacent residues.

Amino acids may be referred to herein by either their commonly known three letter symbols or by the one-letter symbols recommended by the IUPAC-IUB Biochemical Nomenclature Commission.

As to amino acid sequences, one of skill will recognize that individual substitutions, deletions or additions to a peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art.

Macromolecular structures such as polypeptide structures can be described in terms of various levels of organization. For a general discussion of this organization, see, e.g., Alberts et al., Molecular Biology of the Cell (3rd ed., 1994) and Cantor and Schimmel, Biophysical Chemistry Part I. The Conformation of Biological Macromolecules (1980). “Primary structure” refers to the amino acid sequence of a particular peptide. “Secondary structure” refers to locally ordered, three dimensional structures within a polypeptide. These structures are commonly known as domains. Domains are portions of a polypeptide that form a compact unit of the polypeptide and are typically 50 to 350 amino acids long. Typical domains are made up of sections of lesser organization such as stretches of β-sheet and α-helices. “Tertiary structure” refers to the complete three dimensional structure of a polypeptide monomer. “Quaternary structure” refers to the three dimensional structure formed by the noncovalent association of independent tertiary units. Anisotropic terms are also known as energy terms.

The term “administration” or “administering” includes routes of introducing the compound(s) to a subject to perform their intended function. Examples of routes of administration which can be used include injection (subcutaneous, intravenous, parenterally, intraperitoneally, intrathecal), topical, oral, inhalation, rectal and transdermal.

The term “effective amount” includes an amount effective, at dosages and for periods of time necessary, to achieve the desired result. An effective amount of compound may vary according to factors such as the disease state, age, and weight of the subject, and the ability of the compound to elicit a desired response in the subject. Dosage regimens may be adjusted to provide the optimum therapeutic response. An effective amount is also one in which any toxic or detrimental effects (e.g., side effects) of the elastase inhibitor compound are outweighed by the therapeutically beneficial effects.

The phrases “systemic administration,” “administered systemically”, “peripheral administration” and “administered peripherally” as used herein mean the administration of a compound(s), drug or other material, such that it enters the patient's system and, thus, is subject to metabolism and other like processes.

The term “therapeutically effective amount” refers to that amount of the compound being administered sufficient to prevent development of or alleviate to some extent one or more of the symptoms of the condition or disorder being treated.

A therapeutically effective amount of compound (i.e., an effective dosage) may range from about 0.005 μg/kg to about 200 mg/kg, preferably about 0.1 mg/kg to about 200 mg/kg, more preferably about 10 mg/kg to about 100 mg/kg of body weight. In other embodiments, the therapeutically effect amount may range from about 1.0 pM to about 100 nM. The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of a compound can include a single treatment or, preferably, can include a series of treatments. In one example, a subject is treated with a compound in the range of between about 0.005 μg/kg to about 200 mg/kg of body weight, one time per week for between about 1 to 10 weeks, preferably between 2 to 8 weeks, more preferably between about 3 to 7 weeks, and even more preferably for about 4, 5, or 6 weeks. It will also be appreciated that the effective dosage of a compound used for treatment may increase or decrease over the course of a particular treatment.

The term “chiral” refers to molecules which have the property of non-superimposability of the mirror image partner, while the term “achiral” refers to molecules which are superimposable on their mirror image partner.

The term “diastereomers” refers to stereoisomers with two or more centers of dissymmetry and whose molecules are not mirror images of one another.

The term “enantiomers” refers to two stereoisomers of a compound which are non-superimposable mirror images of one another. An equimolar mixture of two enantiomers is called a “racemic mixture” or a “racemate.”

The term “isomers” or “stereoisomers” refers to compounds which have identical chemical constitution, but differ with regard to the arrangement of the atoms or groups in space.

The term “prodrug” includes compounds with moieties which can be metabolized in vivo. Generally, the prodrugs are metabolized in vivo by esterases or by other mechanisms to active drugs. Examples of prodrugs and their uses are well known in the art (See, e.g., Berge et al. (1977) “Pharmaceutical Salts”, J. Pharm. Sci. 66:1-19). The prodrugs can be prepared in situ during the final isolation and purification of the compounds, or by separately reacting the purified compound in its free acid form or hydroxyl with a suitable esterifying agent. Hydroxyl groups can be converted into esters via treatment with a carboxylic acid. Examples of prodrug moieties include substituted and unsubstituted, branch or unbranched lower alkyl ester moieties, (e.g., propionoic acid esters), lower alkenyl esters, di-lower alkyl-amino lower-alkyl esters (e.g., dimethylaminoethyl ester), acylamino lower alkyl esters (e.g., acetyloxymethyl ester), acyloxy lower alkyl esters (e.g., pivaloyloxymethyl ester), aryl esters (phenyl ester), aryl-lower alkyl esters (e.g., benzyl ester), substituted (e.g., with methyl, halo, or methoxy substituents) aryl and aryl-lower alkyl esters, amides, lower-alkyl amides, di-lower alkyl amides, and hydroxy amides. Preferred prodrug moieties are propionoic acid esters and acyl esters. Prodrugs which are converted to active forms through other mechanisms in vivo are also included. Embodiments of the invention include prodrugs of any of the compounds of the formulae herein.

The term “subject” refers to animals such as mammals, including, but not limited to, primates (e.g., humans), cows, sheep, goats, horses, dogs, cats, rabbits, rats, mice and the like. In certain embodiments, the subject is a human.

Furthermore the compounds of the invention include olefins having either geometry: “Z” refers to what is referred to as a “cis” (same side) conformation whereas “E” refers to what is referred to as a “trans” (opposite side) conformation. With respect to the nomenclature of a chiral center, the terms “d” and “l” configuration are as defined by the IUPAC Recommendations. As to the use of the terms, diastereomer, racemate, epimer and enantiomer, these will be used in their normal context to describe the stereochemistry of preparations.

As used herein, the term “alkyl” refers to a straight-chained or branched hydrocarbon group containing 1 to 12 carbon atoms. The term “lower alkyl” refers to a C1-C6 alkyl chain. Examples of alkyl groups include methyl, ethyl, n-propyl, isopropyl, tert-butyl, and n-pentyl. Alkyl groups may be optionally substituted with one or more substituents.

The term “alkenyl” refers to an unsaturated hydrocarbon chain that may be a straight chain or branched chain, containing 2 to 12 carbon atoms and at least one carbon-carbon double bond. Alkenyl groups may be optionally substituted with one or more substituents.

The term “alkynyl” refers to an unsaturated hydrocarbon chain that may be a straight chain or branched chain, containing the 2 to 12 carbon atoms and at least one carbon-carbon triple bond. Alkynyl groups may be optionally substituted with one or more substituents.

The sp² or sp carbons of an alkenyl group and an alkynyl group, respectively, may optionally be the point of attachment of the alkenyl or alkynyl groups.

The term “alkoxy” refers to an —O-alkyl radical.

As used herein, the term “halogen”, “hal” or “halo” means —F, —Cl, —Br or —I.

The term “cycloalkyl” refers to a hydrocarbon 3-8 membered monocyclic or 7-14 membered bicyclic ring system having at least one saturated ring or having at least one non-aromatic ring, wherein the non-aromatic ring may have some degree of unsaturation. Cycloalkyl groups may be optionally substituted with one or more substituents. In one embodiment, 0, 1, 2, 3, or 4 atoms of each ring of a cycloalkyl group may be substituted by a substituent. Representative examples of cycloalkyl group include cyclopropyl, cyclopentyl, cyclohexyl, cyclobutyl, cycloheptyl, cyclopentenyl, cyclopentadienyl, cyclohexenyl, cyclohexadienyl, and the like.

The term “aryl” refers to a hydrocarbon monocyclic, bicyclic or tricyclic aromatic ring system. Aryl groups may be optionally substituted with one or more substituents. In one embodiment, 0, 1, 2, 3, 4, 5 or 6 atoms of each ring of an aryl group may be substituted by a substituent. Examples of aryl groups include phenyl, naphthyl, anthracenyl, fluorenyl, indenyl, azulenyl, and the like.

The term “heteroaryl” refers to an aromatic 5-8 membered monocyclic, 8-12 membered bicyclic, or 11-14 membered tricyclic ring system having 1-4 ring heteroatoms if monocyclic, 1-6 heteroatoms if bicyclic, or 1-9 heteroatoms if tricyclic, said heteroatoms selected from O, N, or S, and the remainder ring atoms being carbon (with appropriate hydrogen atoms unless otherwise indicated). Heteroaryl groups may be optionally substituted with one or more substituents. In one embodiment, 0, 1, 2, 3, or 4 atoms of each ring of a heteroaryl group may be substituted by a substituent. Examples of heteroaryl groups include pyridyl, furanyl, thienyl, pyrrolyl, oxazolyl, oxadiazolyl, imidazolyl thiazolyl, isoxazolyl, quinolinyl, pyrazolyl, isothiazolyl, pyridazinyl, pyrimidinyl, pyrazinyl, triazinyl, isoquinolinyl, indazolyl, and the like.

The term “heterocycloalkyl” refers to a nonaromatic 3-8 membered monocyclic, 7-12 membered bicyclic, or 10-14 membered tricyclic ring system comprising 1-3 heteroatoms if monocyclic, 1-6 heteroatoms if bicyclic, or 1-9 heteroatoms if tricyclic, said heteroatoms selected from O, N, S, B, P or Si, wherein the nonaromatic ring system is completely saturated. Heterocycloalkyl groups may be optionally substituted with one or more substituents. In one embodiment, 0, 1, 2, 3, or 4 atoms of each ring of a heterocycloalkyl group may be substituted by a substituent. Representative heterocycloalkyl groups include piperidinyl, piperazinyl, tetrahydropyranyl, morpholinyl, thiomorpholinyl, 1,3-dioxolane, tetrahydrofuranyl, tetrahydrothienyl, thiirenyl, and the like.

The term “alkylamino” refers to an amino substituent which is further substituted with one or two alkyl groups. The term “aminoalkyl” refers to an alkyl substituent which is further substituted with one or more amino groups. The term “hydroxyalkyl” or “hydroxylalkyl” refers to an alkyl substituent which is further substituted with one or more hydroxyl groups. The alkyl or aryl portion of alkylamino, aminoalkyl, mercaptoalkyl, hydroxyalkyl, mercaptoalkoxy, sulfonylalkyl, sulfonylaryl, alkylcarbonyl, and alkylcarbonylalkyl may be optionally substituted with one or more substituents.

Acids and bases useful in the methods herein are known in the art. Acid catalysts are any acidic chemical, which can be inorganic (e.g., hydrochloric, sulfuric, nitric acids, aluminum trichloride) or organic (e.g., camphorsulfonic acid, p-toluenesulfonic acid, acetic acid, ytterbium triflate) in nature. Acids are useful in either catalytic or stoichiometric amounts to facilitate chemical reactions. Bases are any basic chemical, which can be inorganic (e.g., sodium bicarbonate, potassium hydroxide) or organic (e.g., triethylamine, pyridine) in nature. Bases are useful in either catalytic or stoichiometric amounts to facilitate chemical reactions.

Alkylating agents are any reagent that is capable of effecting the alkylation of the functional group at issue (e.g., oxygen atom of an alcohol, nitrogen atom of an amino group). Alkylating agents are known in the art, including in the references cited herein, and include alkyl halides (e.g., methyl iodide, benzyl bromide or chloride), alkyl sulfates (e.g., methyl sulfate), or other alkyl group-leaving group combinations known in the art. Leaving groups are any stable species that can detach from a molecule during a reaction (e.g., elimination reaction, substitution reaction) and are known in the art, including in the references cited herein, and include halides (e.g., I—, Cl—, Br—, F—), hydroxy, alkoxy (e.g., —OMe, —O-t-Bu), acyloxy anions (e.g., —OAc, —OC(O)CF₃), sulfonates (e.g., mesyl, tosyl), acetamides (e.g., —NHC(O)Me), carbamates (e.g., N(Me)C(O)Ot-Bu), phosphonates (e.g., —OP(O)(OEt)₂), water or alcohols (protic conditions), and the like.

In certain embodiments, substituents on any group (such as, for example, alkyl, alkenyl, alkynyl, aryl, aralkyl, heteroaryl, heteroaralkyl, cycloalkyl, heterocycloalkyl) can be at any atom of that group, wherein any group that can be substituted (such as, for example, alkyl, alkenyl, alkynyl, aryl, aralkyl, heteroaryl, heteroaralkyl, cycloalkyl, heterocycloalkyl) can be optionally substituted with one or more substituents (which may be the same or different), each replacing a hydrogen atom. Examples of suitable substituents include, but are not limited to alkyl, alkenyl, alkynyl, cycloalkyl, heterocycloalkyl, aralkyl, heteroaralkyl, aryl, heteroaryl, halogen, haloalkyl, cyano, nitro, alkoxy, aryloxy, hydroxyl, hydroxylalkyl, oxo (i.e., carbonyl), carboxyl, formyl, alkylcarbonyl, alkylcarbonylalkyl, alkoxycarbonyl, alkylcarbonyloxy, aryloxycarbonyl, heteroaryloxy, heteroaryloxycarbonyl, thio, mercapto, mercaptoalkyl, arylsulfonyl, amino, aminoalkyl, dialkylamino, alkylcarbonylamino, alkylaminocarbonyl, alkoxycarbonylamino, alkylamino, arylamino, diarylamino, alkylcarbonyl, or arylamino-substituted aryl; arylalkylamino, aralkylaminocarbonyl, amido, alkylaminosulfonyl, arylaminosulfonyl, dialkylaminosulfonyl, alkylsulfonylamino, arylsulfonylamino, imino, carbamido, carbamyl, thioureido, thiocyanato, sulfoamido, sulfonylalkyl, sulfonylaryl, or mercaptoalkoxy.

### Compounds of the Invention and Structure Elucidation

In one aspect, the invention provides a compound according to Formula I:

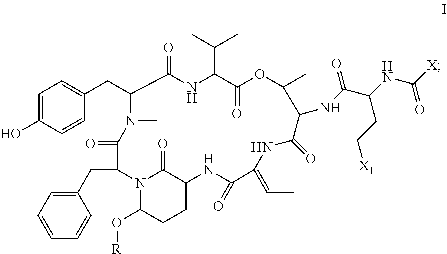

wherein:

R is H or optionally substituted alkyl;

X₁ is optionally substituted aryl, optionally substituted heteroaryl, optionally substituted cycloalkyl, optionally substituted heterocycloalkyl, —OR^{a}, —NR^{a}R^{a}, —C(O)R^{a}, or —OC(O)R^{a};

R^{a}, for each instance is independently selected from H, an optionally substituted alkyl, an optionally substituted cycloalkyl, an optionally substituted heterocycloalkyl, an optionally substituted aryl, an optionally substituted heteroaryl, haloalkyl, hydroxylalkyl, amino, or mono- or di-substituted amine;

X is alkyl or

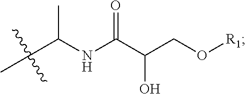

R¹ is selected from H, —S(O)_{q}R^{b}, optionally substituted alkyl, optionally substituted carbocyclic aryl, optionally substituted heteroaryl, optionally substituted cycloalkyl, or optionally substituted heterocyclic;

R^{b} is H, Na, or K;

q is an integer from 0, 1, 2 or 3;

and pharmaceutically acceptable salts, solvate, or hydrate thereof.

In one embodiment, the invention provides a compound of formula I, wherein X is

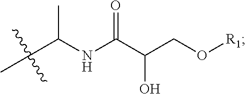

and

R¹ is selected from H, —S(O)_{q}R^{b}, optionally substituted alkyl, optionally substituted carbocyclic aryl, optionally substituted heteroaryl, optionally substituted cycloalkyl, or optionally substituted heteroalicyclic.

In a further embodiment, the invention provides a compound wherein R¹ is H, SO₃H, or SO₃Na.

In another embodiment, the invention provides a compound of formula I, wherein X is alkyl.

In a further embodiment, the invention provides a compound wherein X is pentyl or propyl.

In certain embodiments, the invention provides a compound of formula I, wherein X₁ is optionally substituted aryl.

In a further embodiment, X₁ is para-hydroxy phenyl.

In another embodiment, the invention provides a compound of formula I, wherein X₁ is —C(O)R^{a}.

In a further embodiment, R^{a} is amino.

In one embodiment, the invention provides a compound of formula I, wherein R is H or methyl.

In certain embodiments, the invention provides a compound of formula I selected from the following:

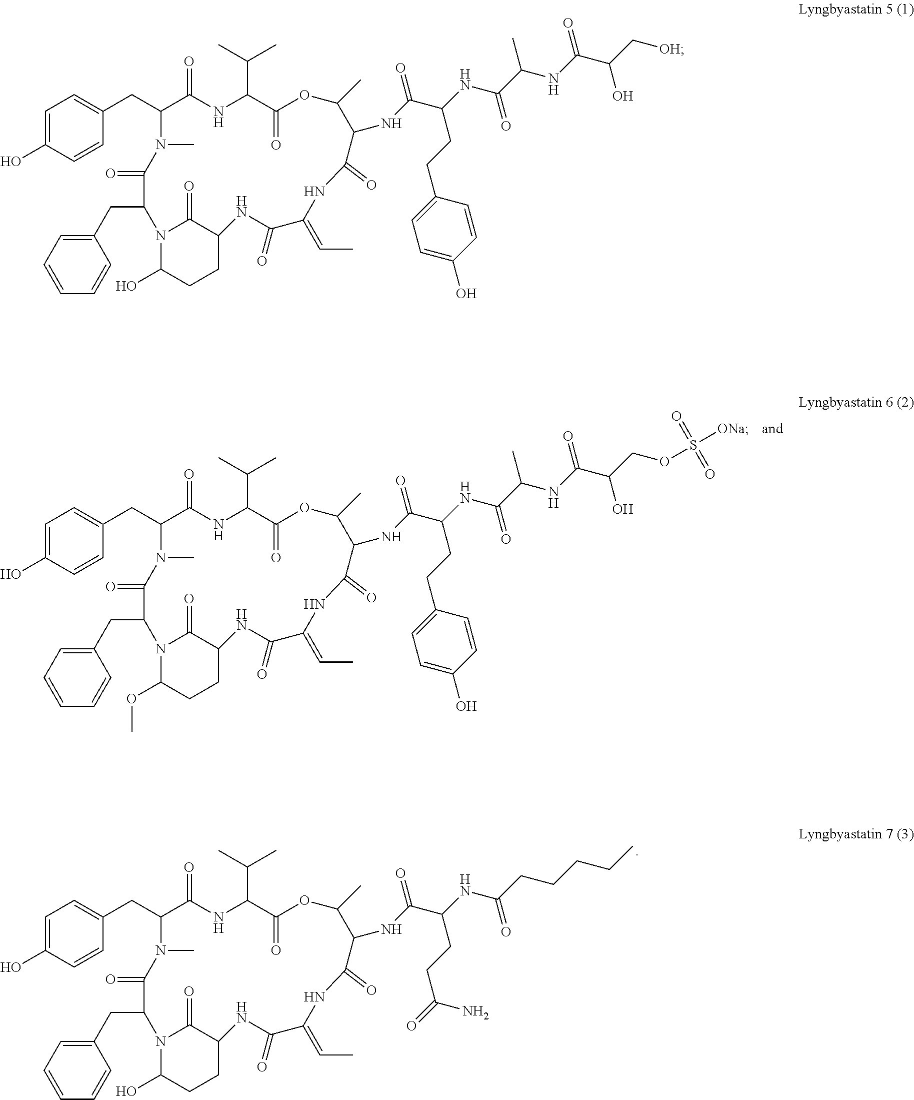

The freeze-dried sample of the lyngbyastatin 4-producing Lyngbya confervoides from reef habitats near Fort Lauderdale, Fla., was extracted with organic solvents, and the extract was partitioned between n-BuOH and H₂O. The n-BuOH layer was fractionated over HP-20 resin and fractions were tested for serine protease inhibitory activities. The active fractions were further chromatographed and subsequently purified by reversed-phase HPLC to afford lyngbyastatin 5 (1) and 6 (2) in submilligram amounts, along with the major metabolite lyngbyastatin 4. Structure determination for 1 and 2 as described below was made possible by using the ultra-sensitive 1-mm triple resonance high-temperature superconducting (HTS) cryogenic probe (Brey, W.; Edison, A. S.; Nast, R. E.; Rocca, J. R.; Saha, S.; Withers, R. S. J. Magn. Reson. 2006, 179, 290-293).

The structure of lyngbyastatin 4 is provided below:

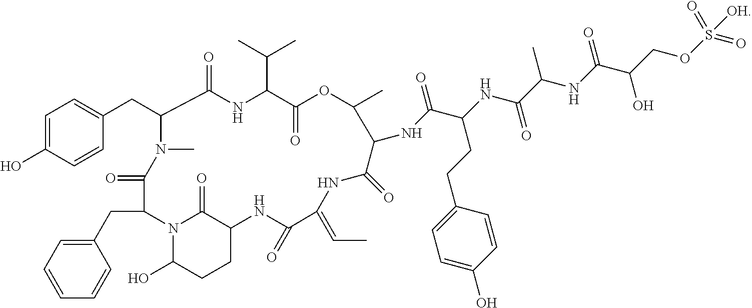

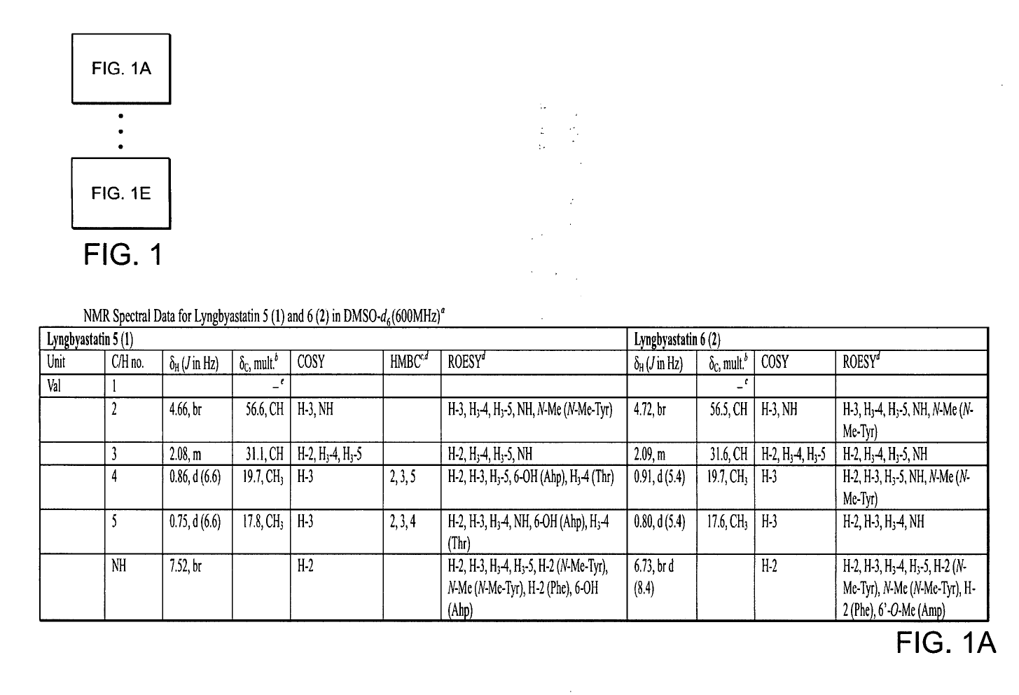

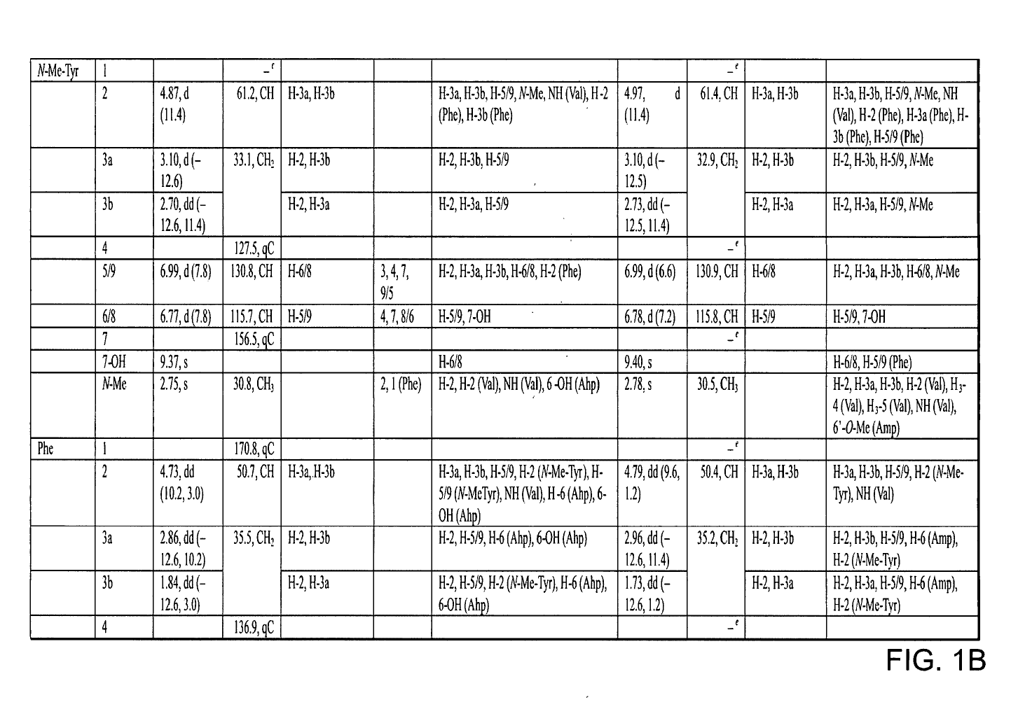

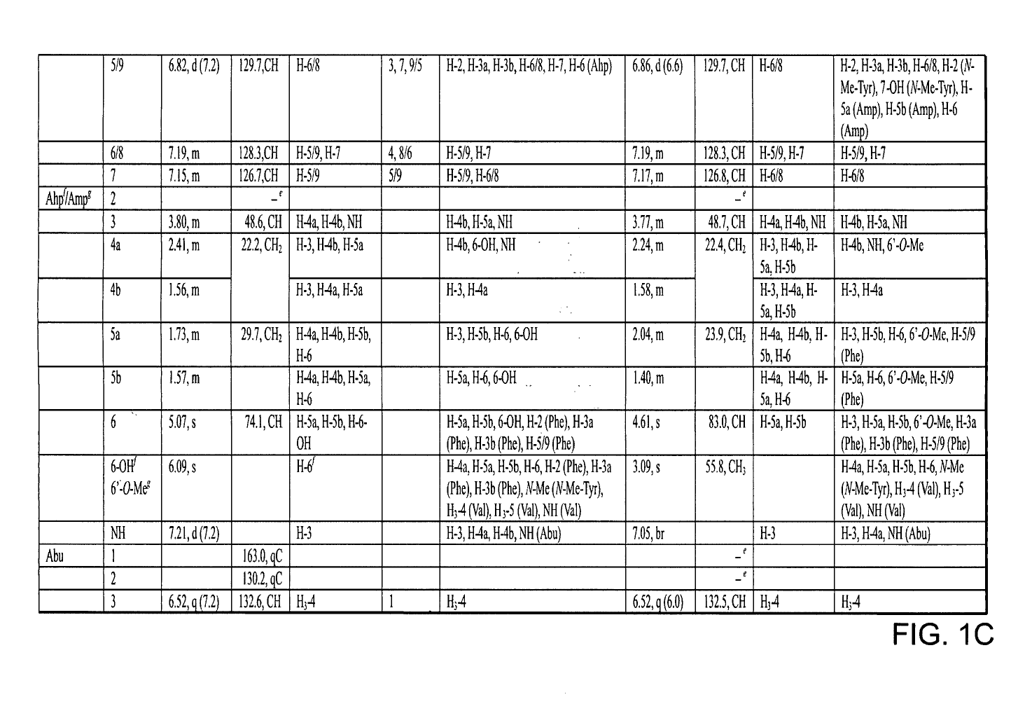

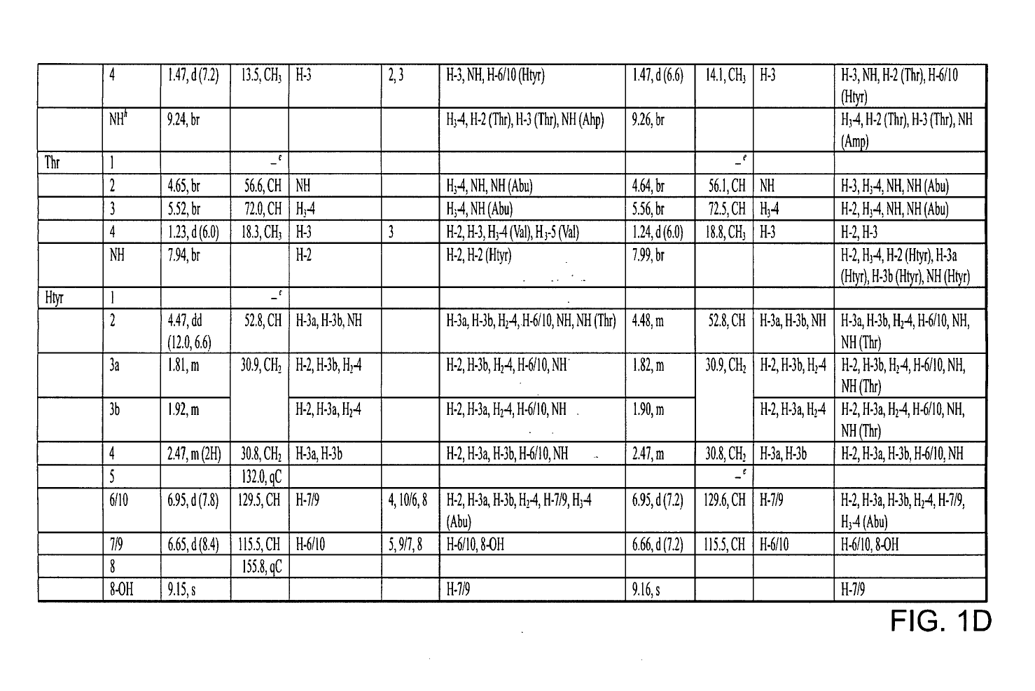

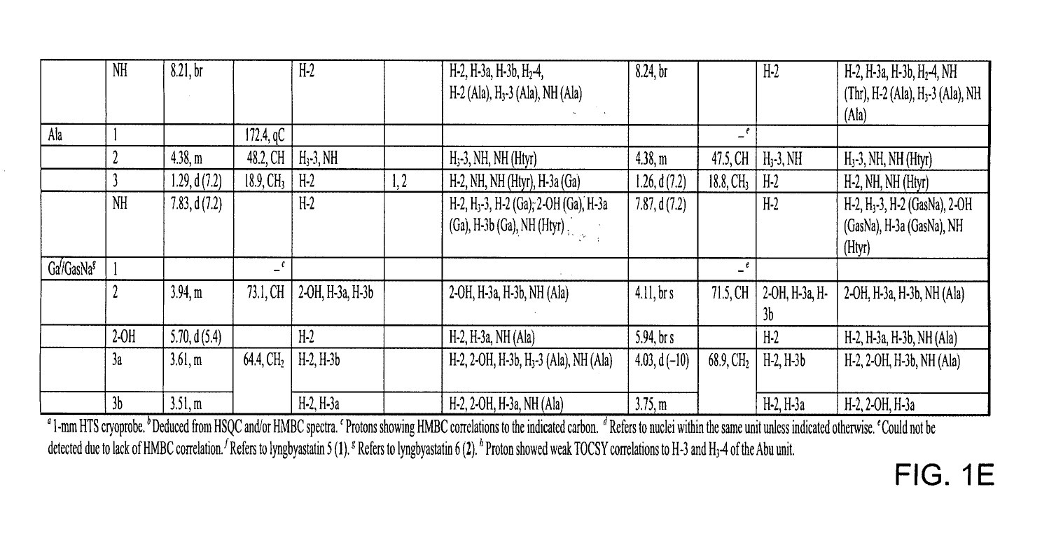

Lyngbyastatin 5 (1) was isolated as a colorless, amorphous solid. NMR data combined with a [M+Na]⁺ peak at m/z 1079.4711 in the HR-ESI/APCI-MS of 1 suggested a molecular formula of C₅₃H₆₈N₈O₁₅. Analysis of the ¹H NMR, COSY, TOCSY, ROESY, HSQC, and HMBC spectra recorded in DMSO-d₆ revealed the presence of alanine, valine, threonine, phenylalanine, N-methyltyrosine, glyceric acid (Ga), homotyrosine (Htyr), 2-amino-2-butenoic acid (Abu) and 3-amino-6-hydroxy-2-piperidone (Ahp) (FIG. 1). ¹H and ¹³C NMR spectral data of 1 closely matched the data reported for lyngbyastatin 4. Despite the lack of many HMBC correlations, sequencing of all amino acid units in 1 was facilitated by ROESY correlations (FIG. 1) that had also been observed for lyngbyastatin 4, supporting the linear sequence of Val-N-Me-Tyr-Phe-Ahp-Abu-Thr-Htyr-Ala-Ga. The cyclized structure for 1 was readily proposed due to the low-field chemical shift of the Thr H-3 (δ_{H} 5.52). Chemical shift differences from NMR data for lyngbyastatin 4 were only apparent for the glyceric acid unit. Compared to H-3 signals in the Ga sulfate (Gas) unit of lyngbyastatin 4, signals for H-3a and H-3b (δ_{H} 3.61 and 3.51) in 1 were shifted upfield (Δδ=0.41 and 0.23 ppm, respectively). This discrepancy suggested that the Ga unit is not sulfated in 1, even though no additional OH signal was observed, presumably due to broadening. This conclusion is consistent with the molecular formula requirements derived from HRMS analysis indicating that compound 1 lacks —SO₃ compared to lyngbyastatin 4. Thus all atoms were accounted for by the proposed structure for 1. To determine if compound 1 was an isolation artifact arising from desulfation of lyngbyastatin 4 during HPLC purification, lyngbyastatin 4 was exposed to TFA to mimic isolation conditions. Repeated HPLC analysis (elution with 0.5% aqueous TFA in 75% MeOH followed by solvent removal under N₂) yielded a single peak corresponding to lyngbyastatin 4, suggesting that lyngbyastatin 5 (1) is indeed a natural product.

The same extract afforded lyngbyastatin 6 (2) as a colorless solid. The molecular formula of 2 was deduced as C₅₄H₆₉N₈O₁₈SNa by HR-ESI/APCI-MS ([M+Na]⁺ at m/z 1195.4257) and NMR spectral data (FIG. 1), suggesting a Na salt. NMR analysis revealed that 2 was a close analog of compound 1, with the same amino acid and hydroxy acid composition. The ¹H NMR, COSY, TOCSY, ROESY and HSQC spectral data were almost identical to those of lyngbyastatin 4, with the exception of the lack of the 6-OH signal of the Ahp unit and an extra signal corresponding to an O-methyl group (δ_{H} 3.09 s, δ_{C} 55.8), accounting for the additional carbon in 2 according to HRMS. Furthermore, the signal for H-6 of this residue (δ_{H} 4.61) was shifted upfield by 0.46 ppm and the corresponding C-6 (δ_{C} 83.0) was shifted downfield by 8.9 ppm compared to 1 (FIG. 1). This NMR data is in agreement with a 3-amino-6-methoxy-2-piperidone (Amp) unit in which the O-Me group has a shielding effect on H-6 and a deshielding effect on C-6, consistent with data for the Amp-containing compound oscillapeptin C. Due to insufficient HMBC correlations owing to scarcity of sample, sequencing of all the amino acid units of 2 was achieved only with the aid of ROESY (FIG. 1). Again, ROESY data ascertained the sequence of Val-N-Me-Tyr-Phe-Amp-Abu-Thr-Htyr-Ala-Ga, and the low-field chemical shift of Thr H-3 (δ_{H} 5.56) allowed us to propose a cyclic depsipeptide structure for 2 rather than a linear peptide. MS data combined with NMR data gave substantial evidence for the presence of the glyceric acid 3′-O-sodium sulfate (GasNa) in the side chain.

A sample of the marine cyanobacterium Lyngbya sp. was collected from a mangrove channel at Summerland Key in the Florida Keys and extracted with organic solvents. Fractionation by solvent partition and successive chromatographic steps using silica, C₁₈ cartridges and finally reversed-phase HPLC afforded lyngbyastatin 7 (3) along with somamide B (4). The structure of somamide B (4) is provided below:

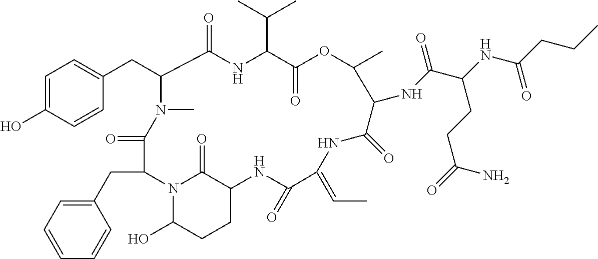

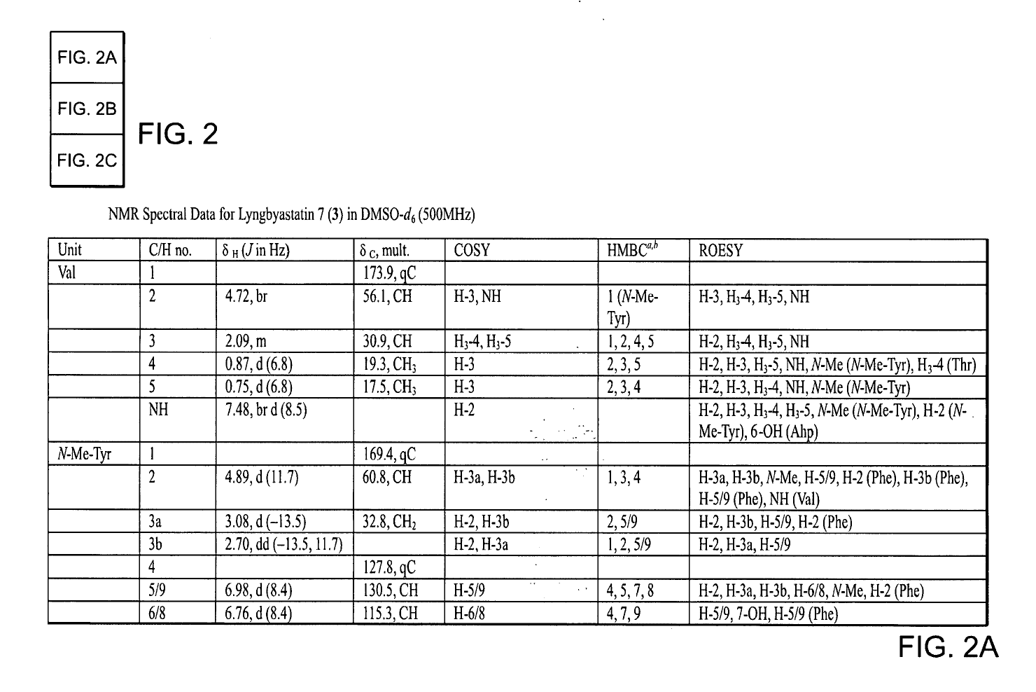

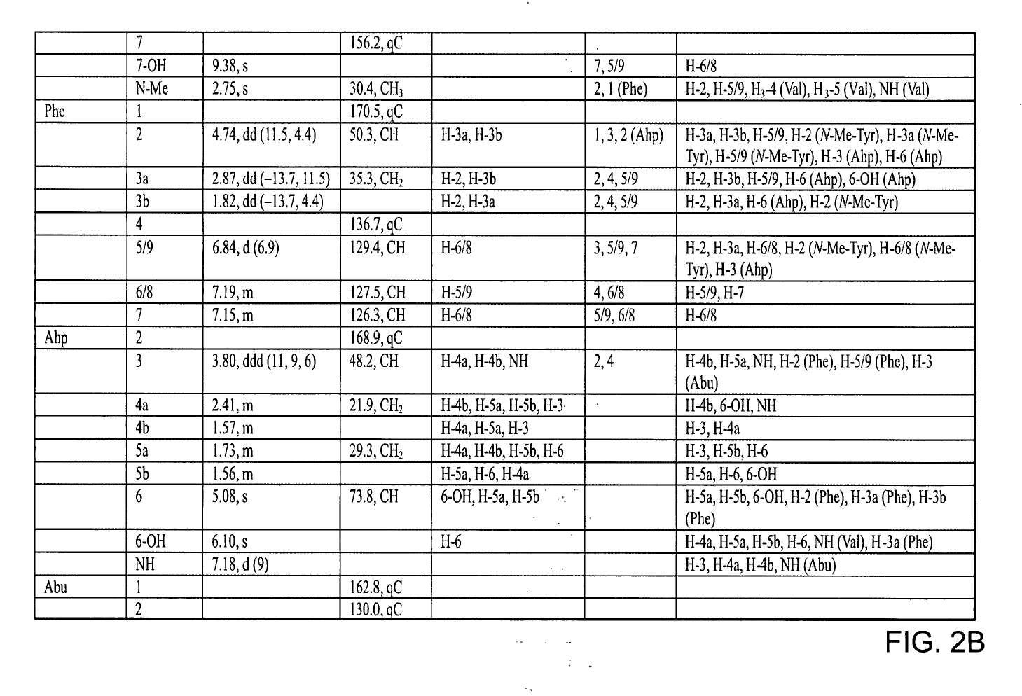

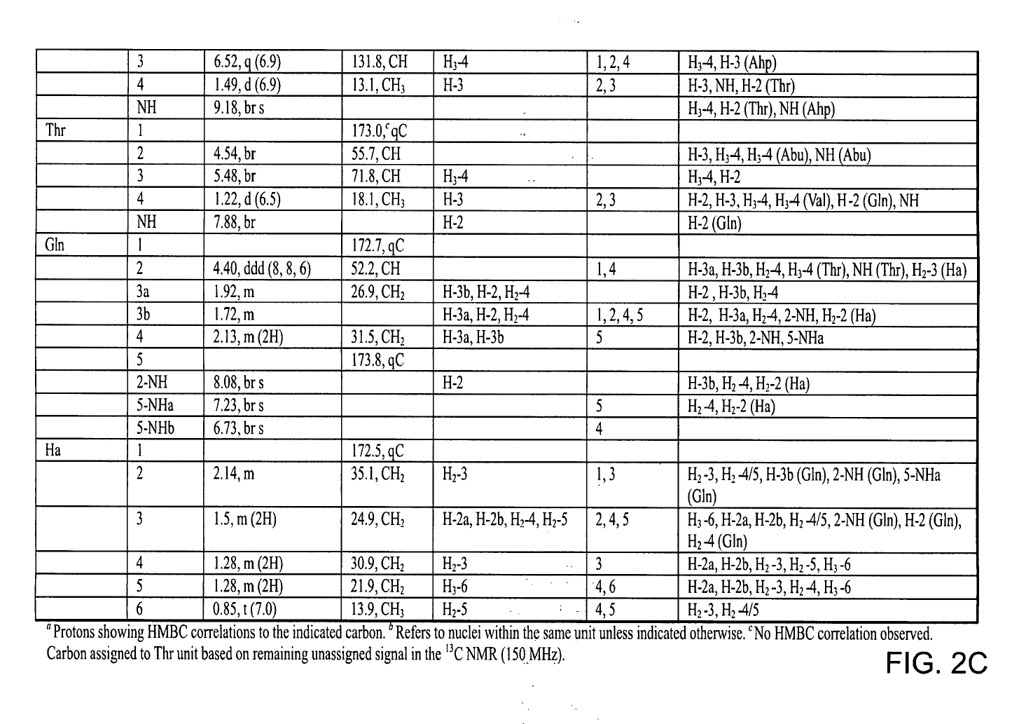

Compound 3 was isolated as a colorless, amorphous solid. NMR data combined with a [M+Na]⁺ peak at m/z 969.4710 in the HR-ESI/APCI-MS of 3 suggested a molecular formula of C₄₈H₆₆N₈O₁₂. Analysis of ¹H NMR, ¹³C NMR, HMQC, COSY, TOCSY, and HMBC spectra revealed the presence of valine, threonine, phenylalanine, N-methyltyrosine, glutamine, hexanoic acid (Ha), Abu and Ahp moieties (FIG. 2). HMBC and ROESY analysis (FIG. 2) and comparison of ¹H and ¹³C NMR data for 1 and 3 revealed that the cyclic core structure for these compounds is identical. Furthermore, ROESY correlations between Thr NH (δ_{H} 7.88) to Gln H-2 (δ_{H} 4.40) and from Gln 2-NH (δ_{H} 8.08) to Ha H₂-2 (δ_{H} 2.14) are consistent with the proposed structure shown for 3. Compound 3 is most closely related to the previously reported cyanobacterial metabolite somamide B, which differs from 3 only by the presence of a terminal butanoic acid (Ba) residue in the side chain instead of the hexanoic acid (Ha) residue in 3. In fact, our further chemical investigation of the lyngbyastatin 7-containing extract also yielded somamide B (4); however, whether or not the absolute configurations for our and the published compound are identical was still unknown up to this point.

ROESY cross peaks between the Abu methyl group and the Abu NH in compounds 1-3 unequivocally established the Z geometry of the Abu group. The absolute configuration of the amino acid residues in compounds 1-3 determined by modified Marfey's analysis (Fujii, K.; Ikai, Y.; Mayumi, T.; Oka, H.; Suzuki, M.; Harada, K. I. Anal. Chem. 1997, 69, 3346-3352) suggested that all the amino acids are in the L-form. The absolute configuration at C-3 of each Ahp residue was determined after CrO₃ oxidation and acid hydrolysis. This reaction sequence liberated L-glutamic acid which permitted us to establish the configuration of the Ahp residues as 3S. It had been previously determined for lyngbyastatin 4 that oxidation prior to hydrolysis increases the yield of phenylalanine (Matthew, S.; Ross, C.; Rocca, J. R; Paul, V. J; Luesch, H. J. Nat. Prod. 2007, 70, 124-127); this procedure again enabled us to clearly assign the 2S configuration to each Phe residue in 1-3. Proton-proton coupling constants and ROESY correlations within the Ahp residues of 1-3 (FIGS. 1 and 2) suggested that the relative configuration and conformation of the Ahp moieties are identical to the one in symplostatin 2, somamide A and lyngbyastatin 4 (3S,6R). For 1-3 the ¹³C NMR and ¹H NMR chemical shifts are equivalent to those reported for somamide B (Nogle, L. M.; Williamson, R. T.; Gerwick, W. H. J. Nat. Prod. 2001, 64, 716-719), suggesting that their relative configurations are identical and thus that these compounds are not diastereomers. And although reliable detection of an optical rotation for 4 was not available, the fact that lyngbyastatin 7 (3) and compound 4 had the same absolute configuration based on Marfey's analysis and that optical rotation data for lyngbyastatin 7 (3) matched closely the data reported for somamide B indicated that compound 4 is indeed somamide B itself but not an enantiomer.

Cyanobacterium Lyngbya sp. was collected from Kemp Channel, a mangrove channel to the southwest of Summerland Key in the Florida Keys. The sample was freeze dried and extracted with CH₂Cl₂-MeOH (1:1). This extract was partitioned with organic solvents followed by various chromatographic steps using silica and C₁₈ and ultimately reversed-phase HPLC to yield compounds 5 and 6.

Kempopeptin A (5) was obtained as a colorless, amorphous solid and shown to have the molecular formula of C₅₀H₇₀N₈O₁₃ as determined by HRESI/APCIMS based on a [M+Na]⁺ peak at m/z of 1013.4965 (calcd for C₅₀H₇₀N₈O₁₃Na, 1013.4960). The presence of a peptide backbone was evident from the ¹H NMR spectrum recorded in DMSO-d₆ due to a tertiary amide N-Me 3H singlet at δ 2.75 and characteristic secondary amide NH resonances occurring as one 1H doublet at δ 7.06 and eight 0.5H doublets at δ 7.42-8.40. The differential integration was suggestive of conformers in only one part of the molecule (Table 3). The combination of ¹H and ¹³C NMR, COSY, HMQC, HMBC, and TOCSY data revealed the presence of valine, N-methyltyrosine, phenylalanine, leucine, proline, two threonine residues, the modified amino acid 3-amino-6-hydroxy-2-piperidone (Ahp) and an acetyl group, with signal doubling for the two threonine moieties, proline, the acetyl group and exchangeable protons of valine and leucine residues. HMBC analysis established the sequence including the planar structure depicted for 5. The doubling of the ¹H NMR signals in the side chain was attributed to restricted rotation around the N-acetyl prolyl amide bond based on ROESY cross-peaks between H-2 of proline (δ_{H} 4.52) and the acetyl protons (δ_{H} 1.83) for the cis isomer and between H-5b of proline (δ_{H} 3.47) and the acetyl protons (δ_{H} 1.95) in the trans isomer. A 1:1 ratio of cis and trans isomers in DMSO-d₆ around the N-acetyl-prolyl bond was also reported for the most closely related metabolite, oscillapeptilide 97-B (Fujii, K.; Sivonen, K.; Naganawa, E.; Harada, K. Tetrahedron 2000, 56, 725-733), which contains an isoleucine instead of the valine in the cyclic core and a glutamine rather than the threonine-2 residue in the side chain.

Acid hydrolysis followed by modified Marfey's analysis (Marfey, P. Carlsberg Res. Commun. 1984, 49, 591-596) established L-configuration of all amino acid residues, while deciphering the Ahp configuration (3S,6R) required prior CrO₃ oxidation, and additionally ROESY analysis of the intact molecule (Table 3) as previously described. (See, Matthew, S.; Ross, C.; Rocca, J. R; Paul, V. J; Luesch, H. J. Nat. Prod. 2007, 70, 124-127; Taori, K.; Matthew, S.; Rocca, J. R.; Paul, V. J.; Luesch, H. J. Nat. Prod. 2007, 70, 1593-1600). The assignment was also consistent with the nearly identical NMR data for kempopeptin A (5) and oscillapeptolide 97-B (Fujii, K.; Sivonen, K.; Naganawa, E.; Harada, K. Tetrahedron 2000, 56, 725-733), suggesting that the relative configuration including conformation of the cyclic core are the same for both compounds.

Kempopeptin B (6) was obtained as a colorless amorphous powder. The HRESI/APCIMS data showed a [M+H]⁺ peak at m/z 993.4663 and an isotope peak of approximately equal intensity at m/z 995.4656, indicating the presence of one bromine atom and a molecular formula of C₄₆H₇₃BrN₈O₁₁ (calcd for C₄₆H₇₄⁷⁹BrN₈O₁₁, 993.4660). Five doublet NH proton signals in the amide range (δ_{H} 7.34, 7.68, 7.80, 7.82, 8.44) and one broad singlet for two primary amide protons (δ_{H} 7.60) in the ¹H NMR spectrum suggested that 6 was a peptide. ¹H NMR, ¹³C NMR, HSQC, COSY and TOCSY analysis revealed seven amino acid spin systems, one carboxylic acid unit, one N-Me (δ_{H} 2.72 s, δ_{C} 30.2) as well as O-Me group (δ_{H} 3.74 s, δ_{C} 56.1), and a 1,3,4-trisubstituted phenyl ring (Table 4). Further NMR including HMBC and ROESY analysis confirmed the presence of two valine units, threonine, isoleucine, lysine, N,O-dimethyl-3′-bromotyrosine, Ahp, and butanoic acid (Ba) moieties, and provided the planar structure for 6 (Table 4). The most unusual structural feature of 6 is arguably the brominated tyrosine residue, which was recently also found in largamides D, F and G, (Plaza, A.; Bewley, C. A. J. Org. Chem. 2006, 71, 6898-6907) symplocamide A (Linington, R. G.; Edwards, D. J.; Shuman, C. F.; McPhail, K. L.; Matainaho, T.; Gerwick, W. H. J. Nat. Prod. 2008, 71, 22-27) and pompanopeptin A (Matthew, S.; Ross, C.; Paul, V. J.; Luesch, H. Tetrahedron 2008, 64, 4081-4089), while other compounds such as scyptolin A (Matern, U.; Oberer, L.; Faichetto, R. A.; Erhard, M.; Konig, W. A.; Herdman, M.; Weckesser, J. Phytochemistry 2001, 58, 1087-1095) and cyanopeptolin 954 (von Elert, E.; Oberer, L.; Merkel, P.; Huhn, T.; Blom, J. F. J. Nat. Prod. 2005, 68, 1324-1327) are chlorinated at this position.

A combination of UV-based Marfey's (Marfey, P. Carlsberg Res. Commun. 1984, 49, 591-596) (Lys, Thr, Val), LC-MS based advanced Marfey's ((a) Fujii, K.; Ikai, Y.; Mayumi, T.; Oka, H.; Suzuki, M.; Harada, K. I. Anal. Chem. 1997, 69, 3346-3352. (b) Fujii, K.; Ikai, Y.; Oka, H.; Suzuki, M.; Harada, K.-I. Anal. Chem. 1997, 69, 5146-5151.) (N,O-diMe-Br-Tyr) and chiral HPLC (Ile) analysis established the L-configuration of these amino acids, while the 3S,6R configuration of the Ahp unit was ascertained as described for 5.

Compounds of the invention can be made by means known in the art of organic synthesis. Methods for optimizing reaction conditions, if necessary minimizing competing by-products, are known in the art. Reaction optimization and scale-up may advantageously utilize high-speed parallel synthesis equipment and computer-controlled microreactors (e.g. Design And Optimization in Organic Synthesis, 2^{nd} Edition, Carlson R, Ed, 2005; Elsevier Science Ltd.; Jähnisch, K et al, Angew. Chem. Int. Ed. Engl. 2004 43: 406; and references therein). Additional reaction schemes and protocols may be determined by the skilled artesian by use of commercially available structure-searchable database software, for instance, SciFinder® (CAS division of the American Chemical Society) and CrossFire Beilstein® (Elsevier MDL), or by appropriate keyword searching using an interne search engine such as Google® or keyword databases such as the US Patent and Trademark Office text database.

The compounds herein may also contain linkages (e.g., carbon-carbon bonds) wherein bond rotation is restricted about that particular linkage, e.g. restriction resulting from the presence of a ring or double bond. Accordingly, all cis/trans and E/Z isomers are expressly included in the present invention. The compounds herein may also be represented in multiple tautomeric forms, in such instances, the invention expressly includes all tautomeric forms of the compounds described herein, even though only a single tautomeric form may be represented. All such isomeric forms of such compounds herein are expressly included in the present invention. All crystal forms and polymorphs of the compounds described herein are expressly included in the present invention. Also embodied are extracts and fractions comprising compounds of the invention. The term isomers is intended to include diastereoisomers, enantiomers, regioisomers, structural isomers, rotational isomers, tautomers, and the like. For compounds which contain one or more stereogenic centers, e.g., chiral compounds, the methods of the invention may be carried out with an enantiomerically enriched compound, a racemate, or a mixture of diastereomers.

Preferred enantiomerically enriched compounds have an enantiomeric excess of 50% or more, more preferably the compound has an enantiomeric excess of 60%, 70%, 80%, 90%, 95%, 98%, or 99% or more. In preferred embodiments, only one enantiomer or diastereomer of a chiral compound of the invention is administered to cells or a subject.

### Methods of Treatment

In one aspect, the invention provides a method of modulating the activity of a protease in a subject, comprising contacting the subject with a compound of formula I (e.g., I, Ia, etc.), in an amount and under conditions sufficient to modulate protease activity.

In another aspect, the invention provides a method of modulating the activity or overactivity of elastase in a subject, comprising contacting the subject with a compound of formula I, in an amount and under conditions sufficient to modulate elastase activity.

In another aspect, the invention provides a method of modulating the activity or overactivity of trypsin in a subject, comprising contacting the subject with a compound of formula I, in an amount and under conditions sufficient to modulate trypsin activity.

In one embodiment, the modulation is inhibition.

In another aspect, the invention provides a method of treating a subject suffering from or susceptible to an elastase overactivity related disorder or disease, comprising administering to the subject an effective amount of a compound or pharmaceutical composition of formula I.

In other aspects, the invention provides a method of treating a subject suffering from or susceptible to an elastase overactivity related disorder or disease, wherein the subject has been identified as in need of treatment for an elastase overactivity related disorder or disease, comprising administering to said subject in need thereof, an effective amount of a compound or pharmaceutical composition of formula I, such that said subject is treated for said disorder.

In other aspects, the invention provides a method of treating a subject suffering from or susceptible to a trypsin overactivity related disorder or disease, wherein the subject has been identified as in need of treatment for a trypsin overactivity related disorder or disease, comprising administering to said subject in need thereof, an effective amount of a compound or pharmaceutical composition of formula I, such that said subject is treated for said disorder.

In certain embodiments, the invention provides a method as described above, wherein the compound of formula I is Lyngbyastatin 5, Lyngbyastatin 6, Lyngbyastatin 7, Kempopeptin A or Kempopeptin B.

In certain embodiments, the invention provides a method of treating a disorder, wherein the disorder is chronic obstructive pulmonary disease (COPD), lung tissue injury, emphysema, hereditary emphysema, rheumatoid arthritis, cystic fibrosis, adult respiratory distress syndrome, reperfusion injury or ischemic-reperfusion injury.

In certain embodiments, the invention provides a method of treating a disorder, wherein the disorder is acute pancreatitis, inflammation or cancer (e.g., angiogenesis related disorders).

In another embodiment, the disorder is an aging-related skin disorder. In a further embodiment, the disorder is wrinkling or cutaneous wrinkling.

In certain embodiments, the subject is a mammal, preferably a primate or human.

In another embodiment, the invention provides a method as described above, wherein the effective amount of the compound of formula I ranges from about 0.005 μg/kg to about 200 mg/kg. In certain embodiments, the effective amount of the compound of formula I ranges from about 0.1 mg/kg to about 200 mg/kg. In a further embodiment, the effective amount of compound of formula I ranges from about 10 mg/kg to 100 mg/kg.

In other embodiments, the invention provides a method as described above wherein the effective amount of the compound of formula I ranges from about 1.0 pM to about 500 nM. In certain embodiments, the effective amount ranges from about 10.0 pM to about 1000 pM. In another embodiment, the effective amount ranges from about 1.0 nM to about 10 nM.

In another embodiment, the invention provides a method as described above, wherein the compound of formula I is administered intravenously, intramuscularly, subcutaneously, intracerebroventricularly, orally or topically.

In other embodiments, the invention provides a method as described above, wherein the compound of formula I is administered alone or in combination with one or more other therapeutics. In a further embodiment, the additional therapeutic agent is an anti-COPD agent, an anti-emphysema agent, or an anti-wrinkle agent.

In another aspect the invention provides a method of treating chronic obstructive pulmonary disease (COPD), lung tissue injury, emphysema, hereditary emphysema, rheumatoid arthritis, cystic fibrosis, adult respiratory distress syndrome, reperfusion injury, ischemic-reperfusion injury, or an aging-related skin disorder, comprising administering to said subject in need thereof, an effective amount of Lyngbyastatin 5, Lyngbyastatin 6, Lyngbyastatin 7, and pharmaceutically acceptable salts thereof.

The inhibitory activity of compounds 1-4 was determined against purified serine proteases, elastase, chymotrypsin, and trypsin, and compared side-by-side with the activity of lyngbyastatin 4 at substrate concentrations near the Kₘ values for each enzyme to allow for better assessment of selectivity. Porcine pancreatic elastase inhibitory activities displayed by compounds 1-4 were similar without statistically significant difference, with IC₅₀ values of 3.2±2.0 nM (1), 3.3±0.8 nM (2), 8.3±5.4 nM (3), and 9.5±5.2 nM (4), which were in the same range as for lyngbyastatin 4 (13.9±3.1 nM). Compared with elastase activity, chymotrypsin activity was less compromised upon enzyme incubation with compounds 1-4, IC₅₀ values being 2.8±0.3 μM (1), 2.5±0.8 μM (2), 2.5±0.2 μM (3), and 4.2±0.5 μM (4). For comparison, lyngbyastatin 4 inhibited chymotrypsin with an IC₅₀ of 4.3±0.8 μM under identical conditions. Expectedly, trypsin activity was unaffected by treatment with compounds 1-4 (up to 30 μM tested), which is consistent with our previous findings for lyngbyastatin 4 (Matthew, S.; Ross, C.; Rocca, J. R; Paul, V. J; Luesch, H. J. Nat. Prod. 2007, 70, 124-127).

There have been numerous publications describing the isolation of related Ahp-containing protease inhibitors from cyanobacteria, which are assumed to be enzyme substrate mimics (Itou, Y.; Ishida, K.; Shin, H. J.; Murakami, M. Tetrahedron 1999, 55, 6871-6882; Ploutno, A.; Shoshan, M.; Carmeli, S. J. Nat. Prod. 2002, 65, 973-978; Yamaki, H.; Sitachitta, N.; Sano, T.; Kaya, K. J. Nat. Prod. 2005, 68, 14-18). In agreement with this assumption, compounds 1-4 inhibited elastase in a competitive manner obliging Michaelis-Menten kinetics. Since the residue between Ahp and Thr units presumably determines the specificity towards certain serine proteases (Yamaki, H.; Sitachitta, N.; Sano, T.; Kaya, K. J. Nat. Prod. 2005, 68, 14-18; Lee, A. Y.; Smitka, T. A.; Bonjouklian, R.; Clardy, J. Chem. Biol. 1994, 1, 113-117; Nakanishi, I.; Kinoshita, T.; Sato, A.; Tada, T. Biopolymers 2000, 53, 434-445; Matern, U.; Schleberger, C.; Jelakovic, S.; Weckesser, J.; Schulz, E. G. Chem. Biol. 2003, 10, 997-1001), the Abu moiety appears to strongly contribute to the observed selectivity for elastase (S1 subsite=recognition pocket) so that the cyclic core structure for 1-4 provides a potent inhibitor. In the co-crystal structure of the Abu-containing bicyclic inhibitor FR901277 bound to porcine pancreatic elastase (Nakanishi, I.; Kinoshita, T.; Sato, A.; Tada, T. Biopolymers 2000, 53, 434-445), it has been previously observed that the ethylidene moiety of Abu is stabilized by CH/π interaction (Nishio, M.; Umezawa, Y.; Hirota, M.; Takeuchi, Y. Tetrahedron 1995, 51, 8665-8701). Such an enzyme-inhibitor interaction may also exist for the monocyclic inhibitors 1-4. Furthermore, for related Ahp-containing protease inhibitors, the carbonyl group of the residue that occupies the S1 enzyme subsite displays hydrogen bonding with NH of Ser-195 of porcine pancreatic elastase. However, the carbonyl moiety of the Abu unit of FR901277 did not form this hydrogen bond (Nakanishi, I.; Kinoshita, T.; Sato, A.; Tada, T. Biopolymers 2000, 53, 434-445). This fact may be attributed to a rigid and coplanar conformation of the backbone atoms due to the carbon-carbon double bond, potentially affecting elastase-inhibitory activity.

The side chain in related inhibitors has been postulated to provide additional interaction points for hydrogen bonding with the enzyme. The Thr unit which forms the ester bond to yield the cyclodepsipeptide core occupies the S2 subsite of the protease. The two consecutive residues located N-terminal to this Thr residue are important determinants for efficient elastase-inhibitor complexes based on co-crystal structures for FR901277 and scyptolin A with the enzyme (S3 and S4 subsites). However, comparable bioassay data for cyclodepsipeptides 1-4 indicate that the corresponding compositional difference in the side chain between 1 and 2 (Htyr-Ala) versus 3 and 4 (Gln-Ha/Ba) is irrelevant and the side chain is overall less influential on the elastase-inhibitory activity. In agreement, scyptolin A (Matern, U.; Schleberger, C.; Jelakovic, S.; Weckesser, J.; Schulz, E. G. Chem. Biol. 2003, 10, 997-1001), planktopeptin BL1125 and planktopeptin BL1061 (Grach-Pogrebinsky, O.; Sedmak, B.; Carmeli, S. Tetrahedron 2003, 59, 8329-8336), all of which contain Leu instead of the Abu unit, display similar activities (IC₅₀s 40-160 nM), although the side chains differ for each compound. Some marginal selectivity for elastase and chymotrypsin was observed among the two planktopeptins. However, planktopeptin BL843 contains only one residue (Glu-γ-lactam) N-terminal to the Thr-Ahp sequence (thus has no residue to occupy the S4 enzyme subsite) and exhibits one order of magnitude lower protease-inhibitory activity. This indicates the requirement of at least two units at these positions for strong activity.

Remarkably, the fact that the protease-inhibitory activity is retained in the O-methylated (Amp) derivative, lyngbyastatin 6 (2), demonstrates that the hydroxyl proton in the Ahp unit is not critical for the inhibition of elastase or chymotrypsin. Inhibitor-enzyme co-crystal structures obtained for related Ahp-containing cyclodepsipeptides also revealed that the OH group of Ahp does not take part in any hydrogen bond formation with the enzyme, but the hydroxyl oxygen atom forms intra- and intermolecular hydrogen bonds with NH of L-Val and a water molecule, respectively (Lee, A. Y.; Smitka, T. A.; Bonjouklian, R.; Clardy, J. Chem. Biol. 1994, 1, 113-117; Nakanishi, I.; Kinoshita, T.; Sato, A.; Tada, T. Biopolymers 2000, 53, 434-445; Matern, U.; Schleberger, C.; Jelakovic, S.; Weckesser, J.; Schulz, E. G. Chem. Biol. 2003, 10, 997-1001). Thus its role as a hydrogen acceptor and its conformation appears unaltered by O-methylation, in agreement with virtually identical NMR data in DMSO-d₆ for lyngbyastatins 4 and 5 (1) versus 6 (2). This is in contrast to activity data reported for the Amp-containing compound oscillapeptin C, which supposedly does not inhibit elastase because of its O-Me group (but still inhibits chymotrypsin) (Itou, Y.; Ishida, K.; Shin, H. J.; Murakami, M. Tetrahedron 1999, 55, 6871-6882).

The inhibitory activity of compounds 5 and 6 against elastase, chymotrypsin and trypsin was determined. Kempopeptin A (1) inhibited elastase with a slight selectivity over chymotrypsin; conversely, kempopeptin B (2) inhibited only trypsin activity (Table 5). These results are in accordance with previous crystallographic and structure-activity relationship data, suggesting that the amino acid residue between Thr and Ahp binds to the enzyme's specificity pocket and thus plays an important role in determining the selectivity towards serine proteases. (Linington, R. G.; Edwards, D. J.; Shuman, C. F.; McPhail, K. L.; Matainaho, T.; Gerwick, W. H. J. Nat. Prod. 2008, 71, 22-27). A hydrophobic amino acid at this position commonly confers preference for chymotrypsin and elastase inhibition (Leu in 5), while a basic amino acid such as lysine or arginine is necessary for trypsin inhibition (Lys in 6). Our IC₅₀ value for 6 against trypsin closely corresponds to data reported for the related lysine-containing metabolite micropeptin SD944 (8.0 μg/mL). See, Reshef, V.; Carmeli, S. Tetrahedron 2001, 57, 2885-2894

The activity of 5 was comparable to those observed for oscillapeptin G (Fujii, K.; Sivonen, K.; Naganawa, E.; Harada, K. Tetrahedron 2000, 56, 725-733), scyptolin A (Matern, U.; Schleberger, C.; Jelakovic, S.; Weckesser, J.; Schulz, E. G. Chem. Biol. 2003, 10, 997-1001), and planktopeptins BL1125 and BL1061, (Grach-Pogrebinsky, O.; Sedmak, B.; Carmeli, S. Tetrahedron 2003, 59, 8329-8336) all of which contain Leu in the cyclic core at this position; however, the Phe residue is replaced by Thr. The different degrees of Tyr modification (chlorination or O-methylation) in these related compounds and substitution of Val for Ile in the planktopeptins likely does not affect protease-inhibitory activity significantly. In lyngbyastatin 7 and somamide B, a 2-amino-2-butenoic acid (Abu) unit presumably occupies the specificity pocket, while all other core residues are the same as in 5. See, Taori, K.; Matthew, S.; Rocca, J. R.; Paul, V. J.; Luesch, H. J. Nat. Prod. 2007, 70, 1593-1600. This allows direct comparison of their activities. Since the side chain composition is less important for activity as long as at least two residues flank the cyclic core (Taori, K.; Matthew, S.; Rocca, J. R.; Paul, V. J.; Luesch, H. J. Nat. Prod. 2007, 70, 1593-1600), the Leu→Abu change within the core structure seems to increase elastase-inhibitory activity but does not enhance chymotrypsin-inhibitory activity (Table 5). A postulated stabilization of the ethylidene moiety by CH/π interaction may be responsible for the potent elastase activity (Nishio, M.; Umezawa, Y.; Hirota, M.; Takeuchi, Y. Tetrahedron 1995, 51, 8665-8701), leading to more pronounced selectivity of lyngbyastatin 7 and somamide B for both proteases compared with 5 (Table 5).

Compound 6 was evaluated for its biological activity against several serine endopeptidases and demonstrated selective in vitro trypsin inhibition when compared to elastase and chymotrypsin inhibitory activities. Trypsin is a proteolytic enzyme that catalyzes the cleavage of peptide bonds on the carboxyl side of either arginine or lysine. The imbalance of trypsin activation within the pancreatic acinar cells presumably leads to the development of acute pancreatitis (Hirota, M.; Ohmuraya, M.; Baba, H. J. Gastroenterol. 2006, 41, 832-836). Additionally, an increase in trypsin activity has been associated with conditions like inflammation and angiogenesis. (Bhattacharya, A.; Smith, G. F.; Cohen, M. L. J. Pharmacol. Exp. Ther. 2001, 297, 573-581).

The effects of compounds 5 and 6 on the proliferation of cancer cells was also assessed. Both compounds did not significantly affect the growth of HT29 colon adenocarcinoma cells at the highest concentration tested (50 μM) by MTT-based cell viability assessment.

### Pharmaceutical Compositions

In one aspect, the invention provides a pharmaceutical composition comprising the compound of formula I and a pharmaceutically acceptable carrier.

In one embodiment, the invention provides a pharmaceutical composition wherein the compound of formula I is Lyngbyastatin 5, Lyngbyastatin 6, Lyngbyastatin 7, Kempopeptin A, or Kempopeptin B, and a pharmaceutically acceptable carrier.

In another embodiment, the invention provides a pharmaceutical composition further comprising an additional therapeutic agent. In a further embodiment, the additional therapeutic agent is an anti-COPD agent, an anti-emphysema agent, or an anti-wrinkle agent. Examples of such agents include: B2 adrenoreceptor agonists (e.g., salbutamol (Ventolin®, Ventodisk®) and terbutaline sulphate (Bricanyl), fenoterol hydrobromide (Berotec®), rimiterol hydrobromide (Pulmadil®), pirbuterol (Exirel®), reproterol hydrochloride (Bronchodil®) and tulobuterol hydrochloride (Brelomax®)); anticholinergic agents (Ipratropium bromide, Atrovent®, and Oxitropium bromide, Oxivent®, (Tiotropium bromide, Ba 679 BR); Methylxanthines including theophylline (Theo-dur®, Phyllocontin®, Uniphyllin®); Corticosteriods including beclomethasone dipropionate (Becotide®, Becloforte®) and budesonide (Pulmicort®), flunisolide inhalation, triamcinolone inhalation, fluticasone inhalation, beclomethasone inhalation, Prednisone, methylprednisolone. Other agents include, for example, Combivent (ipratropium/salbutamol), Advair/Seretide (flucatisone/salmeterol), Symbicort (formoterol/budesonide), Asmanex (mometasone furoate), Foradil, Ariflo (cilomilast), ONO 6126, talnetant, 842470/AWD 12281, IC 485, CP 671305. Non-steroidal anti-inflammatories include, e.g., nedocromil (Tilade). Steroidal anti-inflammatories include, e.g., beclomethasone dipropionate (Aerobec, Beclovent, Beclodisk, Becloforte, Becodisk), budesonide (Pulmicort, Rhinocort), dexamethasone sodium phosophate (Decadron phosphate), flunisolide (Aerobid, Bronalide, Nasalide), fluticasone propionate, triamcinolone acetonide (Azmacort, Nasacort). Anticholinergics include: ipratropium bromide (Atrovent) belladonna alkaloids, Atrovent (ipratropium bromide), atropine, and oxitropium bromide. Antiwrinkle agents include for example, retinoids (e.g., Retin A, retinol), alpha-hydroxyacids, hyaluronic acid, and Botox.

In one aspect, the invention provides a kit comprising an effective amount of a compound of formula I, in unit dosage form, together with instructions for administering the compound to a subject suffering from or susceptible to COPD, emphysema or wrinkling.

The term “pharmaceutically acceptable salts” or “pharmaceutically acceptable carrier” is meant to include salts of the active compounds which are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methancsulfonic, and the like. Also included are salts of amino acids such as arginate and the like, and salts of organic acids like glucuronic or galactunoric acids and the like (see, e.g., Berge et al., Journal of Pharmaceutical Science 66:1-19 (1977)). Certain specific compounds of the present invention contain both basic and acidic functionalities that allow the compounds to be converted into either base or acid addition salts. Other pharmaceutically acceptable carriers known to those of skill in the art are suitable for the present invention.

The neutral forms of the compounds may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

In addition to salt forms, the present invention provides compounds which are in a prodrug form. Prodrugs of the compounds described herein are those compounds that readily undergo chemical changes under physiological conditions to provide the compounds of the present invention. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent.

Certain compounds of the present invention can exist in unsolvated forms as well as solvated forms, including hydrated forms. In general, the solvated forms are equivalent to unsolvated forms and are intended to be encompassed within the scope of the present invention. Certain compounds of the present invention may exist in multiple crystalline or amorphous forms. In general, all physical forms are equivalent for the uses contemplated by the present invention and are intended to be within the scope of the present invention.

The invention also provides a pharmaceutical composition, comprising an effective amount a compound described herein and a pharmaceutically acceptable carrier. In an embodiment, compound is administered to the subject using a pharmaceutically-acceptable formulation, e.g., a pharmaceutically-acceptable formulation that provides sustained delivery of the compound to a subject for at least 12 hours, 24 hours, 36 hours, 48 hours, one week, two weeks, three weeks, or four weeks after the pharmaceutically-acceptable formulation is administered to the subject.

Actual dosage levels and time course of administration of the active ingredients in the pharmaceutical compositions of this invention may be varied so as to obtain an amount of the active ingredient which is effective to achieve the desired therapeutic response for a particular patient, composition, and mode of administration, without being toxic to the patient.

In use, at least one compound according to the present invention is administered in a pharmaceutically effective amount to a subject in need thereof in a pharmaceutical carrier by intravenous, intramuscular, subcutaneous, or intracerebro ventricular injection or by oral administration or topical application. In accordance with the present invention, a compound of the invention may be administered alone or in conjunction with a second, different therapeutic. By “in conjunction with” is meant together, substantially simultaneously or sequentially. In one embodiment, a compound of the invention is administered acutely. The compound of the invention may therefore be administered for a short course of treatment, such as for about 1 day to about 1 week. In another embodiment, the compound of the invention may be administered over a longer period of time to ameliorate chronic disorders, such as, for example, for about one week to several months depending upon the condition to be treated.

By “pharmaceutically effective amount” as used herein is meant an amount of a compound of the invention, high enough to significantly positively modify the condition to be treated but low enough to avoid serious side effects (at a reasonable benefit/risk ratio), within the scope of sound medical judgment. A pharmaceutically effective amount of a compound of the invention will vary with the particular goal to be achieved, the age and physical condition of the patient being treated, the severity of the underlying disease, the duration of treatment, the nature of concurrent therapy and the specific organozinc compound employed. For example, a therapeutically effective amount of a compound of the invention administered to a child or a neonate will be reduced proportionately in accordance with sound medical judgment. The effective amount of a compound of the invention will thus be the minimum amount which will provide the desired effect.

A decided practical advantage of the present invention is that the compound may be administered in a convenient manner such as by intravenous, intramuscular, subcutaneous, oral or intra-cerebroventricular injection routes or by topical application, such as in creams or gels, e.g., in a sunscreen formulation. Depending on the route of administration, the active ingredients which comprise a compound of the invention may be required to be coated in a material to protect the compound from the action of enzymes, acids and other natural conditions which may inactivate the compound. In order to administer a compound of the invention by other than parenteral administration, the compound can be coated by, or administered with, a material to prevent inactivation.

The compound may be administered parenterally or intraperitoneally. Dispersions can also be prepared, for example, in glycerol, liquid polyethylene glycols, and mixtures thereof, and in oils.

The pharmaceutical forms suitable for injectable use include sterile aqueous solutions (where water soluble) or dispersions and sterile powders for the extemporaneous preparation of sterile injectable solutions or dispersions. In all cases the form must be sterile and must be fluid to the extent that easy syringability exists. It must be stable under the conditions of manufacture and storage. The carrier can be a solvent or dispersion medium containing, for example, water, DMSO, ethanol, polyol (for example, glycerol, propylene glycol, liquid polyethylene glycol, and the like), suitable mixtures thereof and vegetable oils. The proper fluidity can be maintained, for example, by the use of a coating such as lecithin, by the maintenance of the required particle size in the case of dispersion. In many cases it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the compound of the invention in the required amount in the appropriate solvent with various of the other ingredients enumerated above, as required, followed by filtered sterilization. Generally, dispersions are prepared by incorporating the various sterilized compounds into a sterile vehicle which contains the basic dispersion medium and the required other ingredients from those enumerated above. In the case of sterile powders for the preparation of sterile injectable solutions, the preferred methods of preparation are vacuum-drying and the freeze-drying technique which yields a powder of the active ingredient plus any additional desired ingredient from previously sterile-filtered solution thereof.

For oral therapeutic administration, the compound may be incorporated with excipients and used in the form of ingestible tablets, buccal tablets, troches, capsules, elixirs, suspensions, syrups, wafers, and the like. Compositions or preparations according to the present invention are prepared so that an oral dosage unit form contains compound concentration sufficient to treat a disorder in a subject.

Some examples of substances which can serve as pharmaceutical carriers are sugars, such as lactose, glucose and sucrose; starches such as corn starch and potato starch; cellulose and its derivatives such as sodium carboxymethycellulose, ethylcellulose and cellulose acetates; powdered tragancanth; malt; gelatin; talc; stearic acids; magnesium stearate; calcium sulfate; vegetable oils, such as peanut oils, cotton seed oil, sesame oil, olive oil, corn oil and oil of theobroma; polyols such as propylene glycol, glycerine, sorbitol, manitol, and polyethylene glycol; agar; alginic acids; pyrogen-free water; isotonic saline; and phosphate buffer solution; skim milk powder; as well as other non-toxic compatible substances used in pharmaceutical formulations such as Vitamin C, estrogen and echinacea, for example. Wetting agents and lubricants such as sodium lauryl sulfate, as well as coloring agents, flavoring agents, lubricants, excipients, tableting agents, stabilizers, anti-oxidants and preservatives, can also be present.

The recitation of a listing of chemical groups in any definition of a variable herein includes definitions of that variable as any single group or combination of listed groups. The recitation of an embodiment for a variable herein includes that embodiment as any single embodiment or in combination with any other embodiments or portions thereof.

### EXAMPLES

The present invention will now be demonstrated using specific examples that are not to be construed as limiting.

### General Experimental Procedures

Optical rotations were measured on a Perkin-Elmer 341 polarimeter. UV spectra were recorded on SpectraMax M5 Molecular Devices. ¹H, ¹³C and 2D NMR spectra were recorded in DMSO-d₆ either on a Bruker Avance 500 MHz or 600 MHz, or Bruker Avance II 600 MHz spectrometer equipped with a 1-mm triple resonance high-temperature superconducting cryogenic probe using residual solvent signals (δ_{H} 2.50 ppm, δ_{C} 39.5 ppm) as internal standards. HMQC and HSQC experiments were optimized for ¹J_{CH}=145 Hz, and HMBC experiments were optimized for ⁿJ_{C,H}=7 Hz. HRMS data were obtained using an Agilent LC-TOF mass spectrometer equipped with an APCI/ESI multimode ion source detector.

### Example 1

### Extraction and Isolation

Samples of Lyngbya confervoides (Paul, V. J.; Thacker, R. W.; Banks, K.; Golubic, S. Coral Reefs 2005, 24, 693-697) were collected off the coast of Fort Lauderdale, Fla. (26°05.9902′ N, 80°05.0184′ W) at a depth of 15 meters in August 2005. A voucher specimen is retained at the Smithsonian Marine Station. The freeze-dried organism was extracted with EtOAc-MeOH (1:1) to afford a crude lipophilic extract which was then partitioned between n-BuOH and H₂O. The n-BuOH extract (6.3 g) was applied to a diaion HP-20 polymeric resin and subsequently fractionated with water and increasing concentrations of MeOH, and then with MeCN. The fraction eluting with 75% aqueous MeOH (175 mg) was subjected to preparative reversed-phase HPLC (LUNA-C18,10u, 100×21.20 mm, 10.0 mL/min; UV detection at 220 and 240 nm) using a MeOH—H₂O linear gradient (30-100% over 40 min and then 100% MeOH for 10 min). Fractions eluting between t_{R} 12-20 min were then repeatedly subjected to semi-preparative reversed-phase HPLC YMC-Pack ODS-AQ, 250×10 mm, 2.0 mL/min; UV detection at 220 and 240 nm) using a linear gradient of 0.5% aqueous TFA in MeOH (60-90% for 25 min, then 90-100% for 10 min and finally 100% MeOH for 10 min) to afford lyngbyastatin 5 (1), t_{R} 13.7 min (0.47 mg), and lyngbyastatin 6 (2), t_{R} 15.0 min (0.17 mg), along with known lyngbyastatin 4, t_{R} 12.2 min (9.6 mg) as the most potent elastase inhibitors in the sample.

Lyngbya sp. was collected from a mangrove channel at the northern end of Summerland Key, Florida Keys (24°39.730′ N, 81°27.791′ W) in May 2006. A voucher specimen is retained at the Smithsonian Marine Station. The freeze-dried sample was extracted with CH₂Cl₂-MeOH (1:1). The resulting lipophilic extract (24.1 g) was partitioned between hexanes and 20% aqueous MeOH, the methanolic phase was evaporated to dryness and the residue further partitioned between n-BuOH and H₂O. The n-BuOH layer was concentrated and subjected to chromatography over silica gel using CH₂Cl₂ and increasing gradients of i-PrOH. Consecutive fractions that eluted with 50 and 75% i-PrOH were individually applied to C₁₈ SPE cartridges and elution was initiated with H₂O followed by aqueous solutions containing 25, 50, 75, and 100% MeOH. Both times, the fractions eluting with 75% aqueous MeOH were then purified by semipreparative reversed-phase HPLC YMC-Pack ODS-AQ, 250×10 mm, 2.0 mL/min; UV detection at 220 and 254 nm) using a MeOH—H₂O linear gradient (50-100% for 60 min and then 100% MeOH for 10 min). The fraction that had eluted with 50% i-PrOH from silica gel yielded compound 3, t_{R} 35.2 min (7.4 mg), while the 75% i-PrOH fraction furnished additional amounts of 3 (3.1 mg) and somamide B (4), t_{R} 26.2 min (1.2 mg). Both compounds accounted for the elastase-inhibitory activity of the extract.

Lyngbyastatin 5 (1): colorless, amorphous powder; UV (MeOH) λₘₐₓ (log ε) 210 (4.57), 280 (sh) (3.79) nm; ¹H NMR, ¹³C NMR, COSY, HMBC, and ROESY data, see Table 1; HR-ESI/APCI-MS m/z [M+Na]⁺ 1079.4711 (calcd for C₅₃H₆₈N₈O₁₅Na 1079.4702).

Lyngbyastatin 6 (2): colorless, amorphous powder; UV (MeOH) λₘₐₓ (log ε) 210 (4.48), 280 (sh) (3.65) nm; ¹H NMR, ¹³C NMR, COSY, and ROESY data, see Table 1; HR-ESI/APCI-MS m/z [M+Na]⁺ 1195.4257 (calcd for C₅₄H₆₉N₈O₁₈SNa₂ 1195.4246).

Lyngbyastatin 7 (3): colorless, amorphous powder; [α]²⁰_{D}-7.4 (c 0.27, MeOH); UV (MeOH) λₘₐₓ (log ε) 230 (3.80), 280 (sh) (3.12); IR (film) 3373 (br), 2961, 1733, 1645 (br), 1539, 1446, 1203, 1026 cm⁻¹; ¹H NMR, ¹³C NMR, COSY, HMBC, and ROESY data, see Table 2; HR-ESI/APCI-MS m/z [M+Na]⁺ 969.4710 (calcd for C₄₈H₆₆N₈O₁₂Na 969.4698).

Somamide B (4): colorless, amorphous powder, UV (MeOH) λₘₐₓ (log ε) 230 (3.74), 280 (sh) (3.10) nm; NMR data, see Nogle, L. M.; Williamson, R. T.; Gerwick, W. H. J. Nat. Prod. 2001, 64, 716-719; HR-ESI/APCI-MS m/z [M+Na]⁺ 941.4407 (calcd for C₄₆H₆₂N₈O₁₂Na 941.4385).

Lyngbya sp. was collected from a mangrove channel at the northern end of Kemp Channel near Summerland Keys (Florida Keys, USA) in May 2006. A morphological characterization including cell measurements was provided with our report of the isolation of lyngbyastatin 7 and somamide B from the same organism. See, Taori, K.; Matthew, S.; Rocca, J. R.; Paul, V. J.; Luesch, H. J. Nat. Prod. 2007, 70, 1593-1600. A specimen preserved in formalin has been retained at the Smithsonian Marine Station.

The freeze dried organism was extracted with CH₂Cl₂-MeOH (1:1). The resulting lipophilic extract (24.1 g) was partitioned between hexanes and 20% aq MeOH, the methanolic phase was evaporated to dryness and the residue further partitioned between n-BuOH and H₂O. The n-BuOH layer was concentrated and subjected to chromatography over silica gel using CH₂Cl₂ and increasing gradients of i-PrOH (2, 5, 10, 20, 50 to 100% i-PrOH) followed by 100% MeOH. The fraction that eluted with 50% i-PrOH was then applied to a C₁₈ SPE cartridge and elution initiated with H₂O followed by aqueous solutions containing 25, 50, 75, and 100% MeOH. The fractions eluting with 75% aq MeOH were then subjected to semipreparative reversed-phase HPLC YMC-pack ODS-AQ, 250×10 mm, 2.0 mL/min; UV detection at 220 and 254 nm) using a MeOH—H₂O linear gradient (50-100% for 60 min and then 100% MeOH for 10 min), yielding compound 5, t_{R} 30.2 min (1.0 mg).

The fraction that eluted with 100% MeOH from silica gel was subjected to Si SPE cartridge and elution started with CH₂Cl₂ followed by CH₂Cl₂ mixtures containing 20, 40, 60, and 80% MeOH, and then 100% MeOH. The fraction eluting with 20% methanolic CH₂Cl₂ was then applied to a semipreparative reversed-phase HPLC column (YMC-Pack ODS-AQ, 250×10 mm, 2.0 mL/min; UV detection at 220 and 254 nm) using a MeOH—H₂O (0.05% TFA) linear gradient (60-100% for 40 min and then 100% MeOH for 15 min). The fraction that had eluted with 100% MeOH from silica gel yielded compound 6, t_{R} 25.2 min (1.6 mg).

Kempopeptin A (5): colorless, amorphous powder; [α]²⁰_{D} (c 0.05, MeOH); UV (MeOH) λₘₐₓ (log ε) 210 (3.66), 280 (sh) (2.67); IR (film) 3374 (br), 2958, 2924, 1735, 1655 (br), 1541, 1449, 1257, 1203, 1139 cm⁻¹; ¹H NMR, ¹³C NMR, HMBC, and ROESY data, see Table 3; HRESI/APCIMS m/z [M+Na]⁺ 1013.4965 (calcd for C₅₀H₇₀N₈O₁₃Na, 1013.4960).

Kempopeptin B (6): colorless, amorphous powder; [α]²⁰_{D}-18 (c 0.16, MeOH); UV (MeOH) λₘₐₓ (log ε) 210 (3.80), 280 (sh) (3.12); IR (film) 3356 (br), 2926, 1738, 1736, 1658 (br), 1530, 1442, 1257, 1205, 1139 cm⁻¹; ¹H NMR, ¹³C NMR, COSY, HMBC, and ROESY data, see Table 4; HRESI/APCIMS m/z [M+H]⁺ 993.4663 (calcd for C₄₆H₇₄⁷⁹BrN₈O₁₁, 993.4660), 995.4656 (calcd for C₄₆H₇₄⁸¹BrN₈O₁₁, 995.4640), 1:1 ion cluster.

### Example 2

### Amino Acid Analysis by Modified Marfey's Method

Samples (˜50 μg each) of compounds 1-4 were subjected to acid hydrolysis (6 N HCl) at 110° C. for 24 h. The hydrolyzates were evaporated to dryness, dissolved in H₂O (100 μl), and divided into two equal portions. To one portion 1 M NaHCO₃ (50 μl) and 1% v/v solution of 1-fluoro-2,4-dinitrophenyl-5-L-leucinamide (L-FDLA) in acetone were added and heated at 80° C. for 3 min. The reaction mixture was then cooled, acidified with 2 N HCl (100 μl), dried and dissolved in H₂O-MeCN (1:1). Aliquots were subjected to reversed-phase HPLC (Alltech Alltima HP C18 HL 5μ, 250×4.6 mm, UV detection at 340 nm) using a linear gradient of MeCN in 0.1% (v/v) aqueous TFA (30-70% MeCN over 50 min). The retention times (t_{R}, min) of the derivatized amino acids in the corresponding hydrolyzates of compounds 1-4 matched with those of L-Thr (13.8), L-Val (23.6), L-Phe (28.5), and N-Me-L-Tyr (40.6). HPLC profiles derived from compounds 1 and 2 additionally revealed peaks for derivatives of L-Ala (19.8) and L-Htyr (44.8), while the profiles of compounds 3 and 4 additionally gave peaks for L-Glu (16.2). Here glutamic acid must have derived from glutamine present in 3 and 4. For comparison, the L-FDLA derivatives of the other standard amino acids not detected in the hydrolyzates had the following retention times (t_{R} in min): L-allo-Thr (14.8), D-allo-Thr (16.9), D-Thr (19.1), D-Val (32.5), D-Phe (35.5), N-Me-D-Tyr (42.6), D-Ala (22.3), D-Htyr (48.4), and D-Glu (17.6).

Portions of the hydrolyzates derived from compounds 1 and 2 were also subjected to chiral HPLC analysis (column, Phenomenex Chirex phase 3126 N,S-dioctyl-(D)-penicillamine, 4.60×250 mm, 5 μm; solvents, 2 mM CuSO₄-MeCN (85:15); flow rate 1.0 mL/min; detection at 254 nm), which allowed detection of D-glyceric acid (t_{R} 13.8 min) but not L-glyceric acid (t_{R} of standard, 10.6 min).

CrO₃ oxidations of 1-4 followed by acid hydrolysis were carried out as described (Matthew, S.; Ross, C.; Rocca, J. R; Paul, V. J; Luesch, H. J. Nat. Prod. 2007, 70, 124-127). The resulting hydrolyzates were derivatized with L-FDLA and aliquots subjected to reversed-phase HPLC as above. When compared to the Marfey profiles without prior oxidation, the HPLC profiles for derivatives resulting from compounds 1 and 2 showed one new peak for L-Glu (t_{R} 16.2 min) and one peak with increased intensity for L-Phe (t_{R} 28.5 min). For compounds 3 and 4, both peaks were already present in the original profile; however, they appeared to be larger after oxidation, while the corresponding D-amino acid derivatives were not detected.

Acid Hydrolysis and Amino Acid Analysis by Modified Marfey's Method. Samples (˜100 μg) of compounds 5 and 6 were subjected to acid hydrolysis at 110° C. for 24 h and analyzed using an L-FDLA based Marfey's procedure as described (Marfey, P. Carlsberg Res. Commun. 1984, 49, 591-596). See, Taori, K.; Matthew, S.; Rocca, J. R.; Paul, V. J.; Luesch, H. J. Nat. Prod. 2007, 70, 1593-1600. The retention times (t_{R}, min) of the L-FDLA derivatized amino acids in the hydrolyzate of compound 5 matched those of L-Thr (14.4), L-Val (24.7), L-Phe (29.6), L-Pro (19.7), L-Leu (28.8), and N-Me-L-Tyr (42.1). Conversely, the L-FDLA derivatives (t_{R}, min) of L-allo-Thr (15.6), D-Thr (20.5), D-allo-Thr (17.1), D-Val (33.9), D-Phe (36.7), D-Pro (23.1), D-Leu (39.5), and N-Me-D-Tyr (43.8) were not detected in the hydrolyzate (retention times given for standard amino acids). The retention times (t_{R}, min) of the derivatized amino acids in the hydrolyzate of compound 6 corresponded to those of L-Thr (14.4), L-Val (24.7), L-Lys (40.5), and L-Ile/L-allo-Ile (27.0); the latter had the same retention times, requiring chiral HPLC analysis of the acid hydrolyzate (see below). Peaks for L-FDLA derivatives of the corresponding isomers were not detected (t_{R}, min): L-allo-Thr (15.6), D-Thr (20.5), D-allo-Thr (17.1), D-Val (33.9), D-Lys (42.5), D-Ile/D-allo-Ile (37.2). N,O-diMe-Br-Tyr adducts could not be reliably detected using this UV-based method, so LC-MS was used instead (see below).

Oxidation-Acid Hydrolysis-Marfey's Analysis Sequence for 5 and 6. CrO₃ oxidation of 5 and 6 followed by acid hydrolysis was carried out as described. See, Matthew, S.; Ross, C.; Rocca, J. R; Paul, V. J; Luesch, H. J. Nat. Prod. 2007, 70, 124-127; Taori, K.; Matthew, S.; Rocca, J. R.; Paul, V. J.; Luesch, H. J. Nat. Prod. 2007, 70, 1593-1600. The resulting hydrolyzates were derivatized with L-FDLA and aliquots subjected to reversed-phase HPLC using UV detection as above. When compared to the Marfey profiles without prior oxidation, the HPLC profiles derived from both compounds 5 and 6 showed one new peak for L-Glu (t_{R} 16.8 min), but not D-Glu (t_{R} 17.8 min).

Chiral HPLC Analysis for 6. Due to overlap of L-FDLA adducts of L-Ile and L-allo-Ile during Marfey's analysis, the acid hydrolyzate derived from 6 was subjected to chiral HPLC analysis (column, Phenomenex Chirex phase 3126 N,S-dioctyl-(D)-penicillamine, 4.60×250 mm, 5 μm; solvents, 2 mM CuSO₄ in H₂O-MeCN (95:5) or 2 mM CuSO₄; flow rate 1.0 mL/min; detection at 254 nm). The absolute configuration of Ile in the hydrolyzate of 6 was determined to be L-Ile by direct comparison with the retention times of authentic standards, while the configurations of the other amino acids obtained from Marfey's analysis were confirmed. The retention times (t_{R}, min) for standard amino acids were as follows: L-Val (16.6), D-Val (21.8), L-Ile (40.8), D-Ile (52.0), L-allo-Ile (34.6), D-allo-Ile (43.1) (solvent mixture 95:5); L-Lys (5.2), D-Lys (6.4), L-Thr (10.8), D-Thr (13.6), L-allo-Thr (15.1), and D-allo-Thr (17.8) (solvent 2 mM CuSO₄).

Advanced Marfey's Analysis of 6. The hydrolyzate of compound 6 was derivatized with L-FDLA and analyzed by LC-MS according to the advanced Marfey's method ((a) Fujii, K.; Ikai, Y.; Mayumi, T.; Oka, H.; Suzuki, M.; Harada, K. I. Anal. Chem. 1997, 69, 3346-3352. (b) Fujii, K.; Ikai, Y.; Oka, H.; Suzuki, M.; Harada, K.-I. Anal. Chem. 1997, 69, 5146-5151) to reveal L-configuration of N,O-diMe-Br-Tyr in 6 as described. See, Matthew, S.; Ross, C.; Paul, V. J.; Luesch, H. Tetrahedron 2008, 64, 4081-4089.

### Example 3

### Protease Inhibition Assays

The test samples for 1-6 were prepared in DMSO by (log/2)-fold dilutions ranging from 1 mM to 100 pM. All assays were performed in triplicate. Phenylmethylsulfonyl fluoride (PMSF) was used as a positive control in the enzyme assays.

To test the inhibition of porcine pancreatic elastase (Elastase-high purity; EPC, EC134), 75 μg/mL solution of elastase was prepared using Tris-HCl (pH 8.0). The Kₘ for elastase was determined to be 1.5 mM for N-succinyl-Ala-Ala-Ala-p-nitroanilide, a concentration which was used subsequently for the inhibitor dose-response experiments. After preincubation of 165 μL of Tris-HCl (pH 8.0), 10 μL of elastase solution, and 10 μL of test samples in DMSO (5% final concentration) in a microtiter plate at 30° C. for 20 min, 15 μL of substrate solution (1.5 mM final concentration) was added to the mixture. The increase in absorbance was measured for 30 min at intervals of 5 min at 405 nm. Competitive binding was determined by plotting enzyme activity against substrate concentrations in the presence of different inhibitor concentrations (Lineweaver-Burk plot).

Inhibitory activity against α-chymotrypsin (bovine pancreas; Sigma, C4129) was determined as follows. A 1-mg/mL solution of chymotrypsin was prepared in assay buffer (50 mM Tris-HCl/100 mM NaCl/1 mM CaCl₂, pH 7.8). After preincubation of 80 μL of assay buffer solution, 10 μL of enzyme solution, and 10 μL of test solution in DMSO in a microtiter plate at 37° C. for 10 min, 50 μL of substrate solution (N-succinyl-Gly-Gly-Phe-p-nitroanilide, 0.75 mM final concentration corresponding to Kₘ) was added to the mixture. The increase in absorbance was measured for 30 min at intervals of 5 min at 405 nm.

Inhibitory activity against trypsin was assayed as described above for chymotrypsin, using trypsin from porcine pancreas (Sigma, T0303) and Nα-benzoyl-DL-arginine-4-nitroanilide hydrochloride as the substrate solution.

### INCORPORATION BY REFERENCE

The contents of all references (including literature references, issued patents, published patent applications, and co-pending patent applications) cited throughout this application are hereby expressly incorporated herein in their entireties by reference.

### EQUIVALENTS

Those skilled in the art will recognize, or be able to ascertain using no more than routine experimentation, many equivalents of the specific embodiments of the invention described herein. Such equivalents are intended with be encompassed by the following claims.

What is claimed:

1. A compound according to Formula Ia: 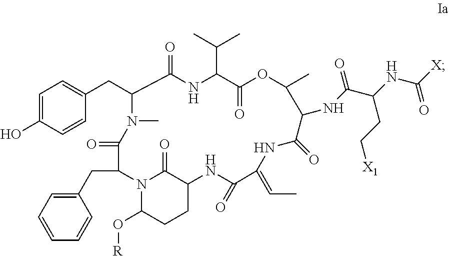
wherein:
R is H or optionally substituted alkyl;
X₁ is optionally substituted aryl, optionally substituted heteroaryl, optionally substituted cycloalkyl, optionally substituted heterocycloalkyl, —OR^{a}, —NR^{a}R^{a}, —C(O)R^{a}, or —OC(O)R^{a};
R^{a}, for each instance is independently selected from H, an optionally substituted alkyl, an optionally substituted cycloalkyl, an optionally substituted heterocycloalkyl, an optionally substituted aryl, an optionally substituted heteroaryl, haloalkyl, hydroxylalkyl, amino, or mono- or di-substituted amine;
X is alkyl or 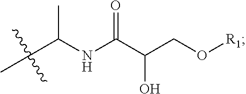
R¹ is selected from H, —S(O)_{q}R^{b}, optionally substituted alkyl, optionally substituted carbocyclic aryl, optionally substituted heteroaryl, optionally substituted cycloalkyl, or optionally substituted heterocyclic;
R^{b} is H, Na, or K;
q is an integer from 0, 1, 2 or 3;
and pharmaceutically acceptable salts, solvate, or hydrate thereof.

2. The compound of formula I, wherein X is 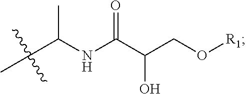
and
R¹ is selected from H, —S(O)_{q}R^{b}, optionally substituted alkyl, optionally substituted carbocyclic aryl, optionally substituted heteroaryl, optionally substituted cycloalkyl, or optionally substituted heteroalicyclic.

3. The compound of claim 2 wherein R¹ is H, SO₃H, or SO₃Na.

4. The compound of claim 1 wherein X is alkyl.

5. The compound of claim 4 wherein X is pentyl or propyl.

6. The compound of claim 1, wherein X₁ is optionally substituted aryl.

7. The compound of claim 6, wherein X₁ is para-hydroxy phenyl.

8. The compound of claim 1, wherein X₁ is —C(O)R^{a}.

9. The compound of claim 8, wherein R^{a} is amino.

10. The compound of claim 1, wherein R is H or methyl.

11. The compound of claim 1 selected from the following:

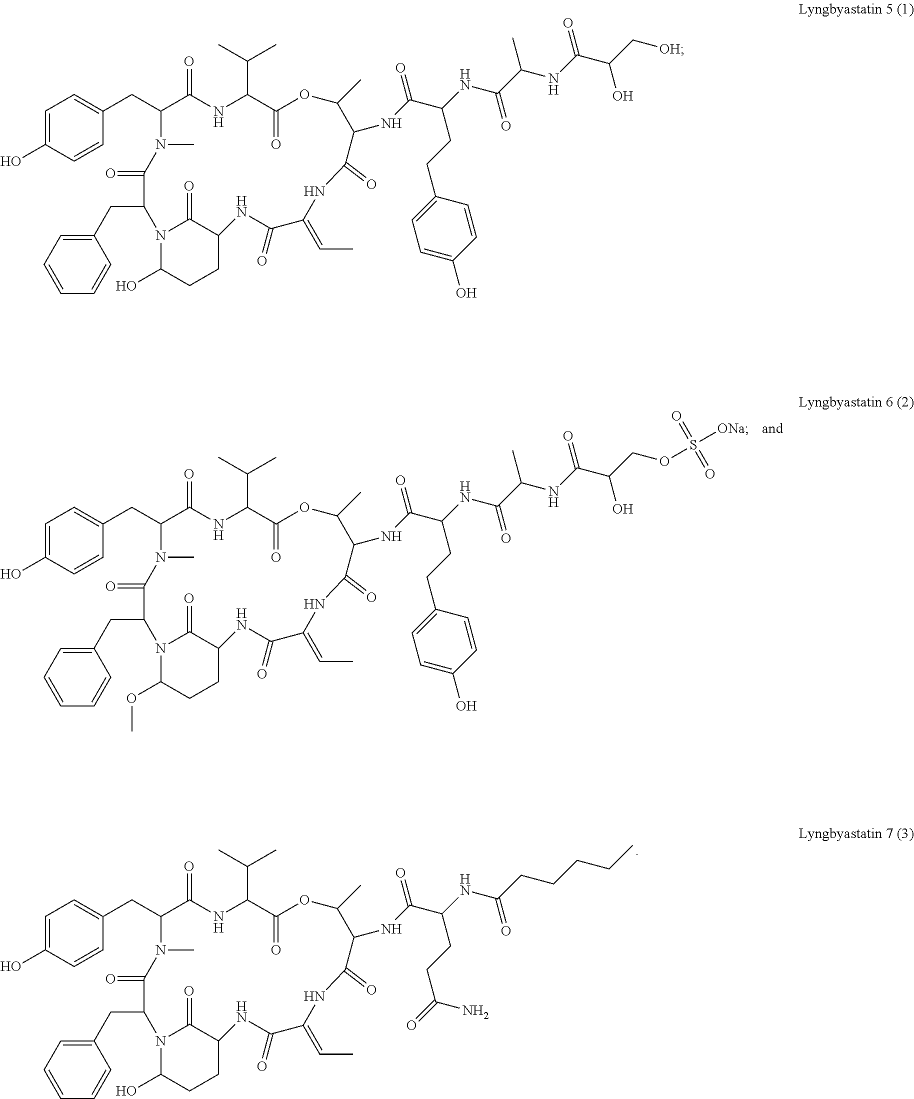

12. A pharmaceutical composition comprising the compound of claim 1 and a pharmaceutically acceptable carrier.

13. The pharmaceutical composition of claim 12, wherein the compound of claim 1 is Lyngbyastatin 5, Lyngbyastatin 6, Lyngbyastatin 7, and a pharmaceutically acceptable carrier.

14. The pharmaceutical composition of claim 12 further comprising an additional therapeutic agent.

15. The pharmaceutical composition of claim 14 wherein the additional therapeutic agent is an anti-COPD agent, an anti-emphysema agent, or an anti-wrinkle agent.

16. A kit comprising an effective amount of a compound of claim 1, in unit dosage form, together with instructions for administering the compound to a subject suffering from or susceptible to COPD, emphysema or wrinkling.

17. A method of modulating the activity of a protease in a subject, comprising contacting the subject with a compound of formula I, in an amount and under conditions sufficient to modulate protease activity.

18. A method of modulating the activity or overactivity of elastase in a subject, comprising contacting the subject with a compound of formula I, in an amount and under conditions sufficient to modulate elastase activity.

19. The method of claim 18, wherein the modulation is inhibition.

20. A method of treating a subject suffering from or susceptible to an elastase overactivity related disorder or disease, comprising administering to the subject an effective amount of a compound or pharmaceutical composition of formula I.

21. A method of treating a subject suffering from or susceptible to an elastase overactivity related disorder or disease, wherein the subject has been identified as in need of treatment for an elastase overactivity related disorder or disease, comprising administering to said subject in need thereof, an effective amount of a compound or pharmaceutical composition of formula I, such that said subject is treated for said disorder.

22. The method of claim 20 or 21, wherein the compound of formula I is Lyngbyastatin 5, Lyngbyastatin 6, or Lyngbyastatin 7.

23. The method of claim 20 or 21, wherein the disorder is chronic obstructive pulmonary disease (COPD), lung tissue injury, emphysema, hereditary emphysema, rheumatoid arthritis, cystic fibrosis, adult respiratory distress syndrome, reperfusion injury or ischemic-reperfusion injury.

24. The method of claim 20 or 21, wherein the disorder is an aging-related skin disorder.

25. The method of claim 24, wherein the disorder is wrinkling or cutaneous wrinkling.

26. The method of claim 20 or 21, wherein the subject is a mammal.

27. The method of claim 26 wherein the subject is a primate or human.

28. The method of claim 20 or 21, wherein the effective amount of the compound of formula I ranges from about 0.005 μg/kg to about 200 mg/kg.

29. The method of claim 28, wherein the effective amount of the compound of formula I ranges from about 0.1 mg/kg to about 200 mg/kg.

30. The method of claim 29, wherein the effective amount of compound of formula I ranges from about 10 mg/kg to 100 mg/kg.

31. The method of claim 20 or 21, wherein the effective amount of the compound of formula I ranges from about 1.0 pM to about 500 nM.

32. The method of claim 20 or 21, wherein the compound of formula I is administered intravenously, intramuscularly, subcutaneously, intracerebroventricularly, orally or topically.

33. The method of claim 20 or 21, wherein the compound of formula I is administered alone or in combination with one or more other therapeutics.

34. The method of claim 33, wherein the additional therapeutic agent is an anti-COPD agent, an anti-emphysema agent, or an anti-wrinkle agent.

35. A method of treating chronic obstructive pulmonary disease (COPD), lung tissue injury, emphysema, hereditary emphysema, rheumatoid arthritis, cystic fibrosis, adult respiratory distress syndrome, reperfusion injury, ischemic-reperfusion injury, or an aging-related skin disorder, comprising administering to said subject in need thereof, an effective amount of Lyngbyastatin 5, Lyngbyastatin 6, Lyngbyastatin 7, or pharmaceutically acceptable salts thereof.

36. A compound according to Formula I: 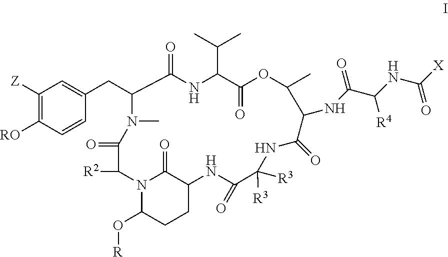
wherein:
each R is independently H or optionally substituted alkyl;
X₁ is optionally substituted aryl, optionally substituted heteroaryl, optionally substituted cycloalkyl, optionally substituted heterocycloalkyl, —OR^{a}, —NR^{a}R^{a}, —C(O)R^{a}, or —OC(O)R^{a};
R^{a}, for each instance is independently selected from H, an optionally substituted alkyl, an optionally substituted cycloalkyl, an optionally substituted heterocycloalkyl, an optionally substituted aryl, an optionally substituted heteroaryl, haloalkyl, hydroxylalkyl, amino, or mono- or di-substituted amine;
X is alkyl, N-acetylpyrrolidin-2-yl, or 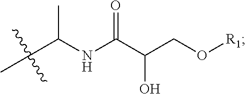
R¹ is selected from H, —S(O)_{q}R^{b}, optionally substituted alkyl, optionally substituted carbocyclic aryl, optionally substituted heteroaryl, optionally substituted cycloalkyl, or optionally substituted heterocyclic;
R² is alkyl, optionally substituted with aryl;
each R³ is independently H, alkyl optionally substituted with NH₂, or both R³ taken together with the carbon to which they are attached form C═CHR;
Each R⁴ is independently alkyl optionally substituted with X₁;
R^{b} is H, Na, or K;
q is an integer from 0, 1, 2 or 3;
each Z is independently H or halogen;
and pharmaceutically acceptable salts, solvate, or hydrate thereof.

37. The compound of claim 36 that is Kempopeptin A or Kempopeptin B.

| | | | | | | | | | | |
|---|---|---|---|---|---|---|---|---|---|---|
| | 5b | 1.57, m | | H-4a, H-4b, H-5a, | | H-5a, H-6, 6-OH | 1.40, m | | H-4a, H-4b, H- | H-5a, H-6, 6′-O—Me, H-5/9 |
| | | | | H-6 | | | | | 5a, H-6 | (Phe) |
| | 6 | 5.07, s | 74.1, CH | H-5a, H-5b, H-6- | | H-5a, H-5b, 6-OH, H-2 (Phe), H-3a | 4.61, s | 83.0, CH | H-5a, H-5b | H-3, H-5a, H-5b, 6′-O—Me, H-3a |
| | | | | OH | | (Phe), H-3b (Phe), H-5/9 (Phe) | | | | (Phe), H-3b (Phe), H-5/9 (Phe) |
| | 6-OH^{f} | 6.09, s | | H-6^{f} | | H-4a, H-5a, H-5b, H-6, H-2 (Phe), H-3a | 3.09, s | 55.8, CH₃ | | H-4a, H-5a, H-5b, H-6, N—Me |
| | 6′-O—Me^{g} | | | | | (Phe), H-3b (Phe), N—Me (N—Me-Tyr), | | | | (N—Me-Tyr), H₃-4 (Val), H₃-5 |
| | | | | | | H₃-4 (Val), H₃-5 (Val), NH (Val) | | | | (Val), NH (Val) |
| | NH | 7.21, d (7.2) | | H-3 | | H-3, H-4a, H-4b, NH (Abu) | 7.05, br | | H-3 | H-3, H-4a, NH (Abu) |
| Abu | 1 | | 163.0, qC | | | | | —^{e} | | |
| | 2 | | 130.2, qC | | | | | —^{e} | | |
| | 3 | 6.52, q (7.2) | 132.6, CH | H₃-4 | 1 | H₃-4 | 6.52, q (6.0) | 132.5, CH | H₃-4 | H₃-4 |
| | 4 | 1.47, d (7.2) | 13.5, CH₃ | H-3 | 2, 3 | H-3, NH, H-6/10 (Htyr) | 1.47, d (6.6) | 14.1, CH₃ | H-3 | H-3, NH, H-2 (Thr), H-6/10 |
| | | | | | | | | | | (Htyr) |
| | NH^{h} | 9.24, br | | | | H₃-4, H-2 (Thr), H-3 (Thr), NH (Ahp) | 9.26, br | | | H₃-4, H-2 (Thr), H-3 (Thr), NH |
| | | | | | | | | | | (Amp) |
| Thr | 1 | | —^{e} | | | | | —^{e} | | |
| | 2 | 4.65, br | 56.6, CH | NH | | H₃-4, NH, NH (Abu) | 4.64, br | 56.1, CH | NH | H-3, H₃-4 NH, NH (Abu) |
| | 3 | 5.52, br | 72.0, CH | H₃-4 | | H₃-4, NH (Abu) | 5.56, br | 72.5, CH | H₃-4 | H-2, H₃-4, NH, NH (Abu) |
| | 4 | 1.23, d (6.0) | 18.3, CH₃ | H-3 | 3 | H-2, H-3, H₃-4 (Val), H₃-5 (Val) | 1.24, d (6.0) | 18.8, CH₃ | H-3 | H-2, H-3 |
| | NH | 7.94, br | | H-2 | | H-2, H-2 (Htyr) | 7.99, br | | | H-2, H₃-4, H-2 (Htyr), H-3a |
| | | | | | | | | | | (Htyr), H-3b (Htyr), NH (Htyr) |
| Htyr | 1 | | —^{e} | | | | | | | |
| | 2 | 4.47, dd | 52.8, CH | H-3a, H-3b, NH | | H-3a, H-3b, H₂-4, H-6/10, NH, NH (Thr) | 4.48, m | 52.8, CH | H-3a, H-3b, NH | H-3a, H-3b, H₂-4, H-6/10, NH, |
| | | (12.0, 6.6) | | | | | | | | NH (Thr) |
| | 3a | 1.81, m | 30.9, CH₂ | H-2, H-3b, H₂-4 | | H-2, H-3b, H₂-4, H-6/10, NH | 1.82, m | 30.9, CH₂ | H-2, H-3b, H₂-4 | H-2, H-3b, H₂-4, H-6/10, NH, |
| | | | | | | | | | | NH (Thr) |
| | 3b | 1.92, m | | H-2, H-3a, H₂-4 | | H-2, H-3a, H₂-4, H-6/10, NH | 1.90, m | | H-2, H-3a, H₂-4 | H-2, H-3a, H₂-4, H-6/10, NH, |
| | | | | | | | | | | NH (Thr) |
| | 4 | 2.47, m (2H) | 30.8, CH₂ | H-3a, H-3b | | H-2, H-3a, H-3b, H-6/10, NH | 2.47, m | 30.8, CH₂ | H-3a, H-3b | H-2, H-3a, H-3b, H-6/10, NH |
| | 5 | | 132.0, qC | | | | | —^{e} | | |
| | 6/10 | 6.95, d (7.8) | 129.5, CH | H-7/9 | 4, 10/6, 8 | H-2, H-3a, H-3b, H₂-4, H-7/9, H₃-4 | 6.95, d (7.2) | 129.6, CH | H-7/9 | H-2, H-3a, H-3b, H₂-4, H-7/9, |
| | | | | | | (Abu) | | | | H₃-4 (Abu) |
| | 7/9 | 6.65, d (8.4) | 115.5, CH | H-6/10 | 5, 9/7, 8 | H-6/10, 8-OH | 6.66, d (7.2) | 115.5, CH | H-6/10 | H-6/10, 8-OH |
| | 8 | | 155.8, qC | | | | | | | |
| | 8-OH | 9.15, s | | | | H-7/9 | 9.16, s | | | H-7/9 |
| | NH | 8.21, br | | H-2 | | H-2, H-3a, H-3b, H₂-4, | 8.24, br | | H-2 | H-2, H-3a, H-3b, H₂-4, NH |
| | | | | | | H-2 (Ala), H₃-3 (Ala), NH (Ala) | | | | (Thr), H-2 (Ala), H₃-3 (Ala), [symbol] |
| | | | | | | | | | | (Ala) |
| Ala | 1 | | 172.4, qC | | | | | —^{e} | | |
| | 2 | 4.38, m | 48.2, CH | H₃-3, NH | | H₃-3, NH, NH (Htyr) | 4.38, m | 47.5, CH | H₃-3, NH | H₃-3, NH, NH (Htyr) |
| | 3 | 1.29, d (7.2) | 18.9, CH₃ | H-2 | 1, 2 | H-2, NH, NH (Htyr), H-3a (Ga) | 1.26, d (7.2) | 18.8, CH₃ | H-2 | H-2, NH, NH (Htyr) |
| | NH | 7.83, d (7.2) | | H-2 | | H-2, H₃-3, H-2 (Ga), 2-OH (Ga), H-3a | 7.87, d (7.2) | | H-2 | H-2, H₃-3, H-2 (GasNa), 2-O [symbol] |
| | | | | | | (Ga), H-3b (Ga), NH (Htyr) | | | | (GasNa), H-3a (GasNa), NH |
| | | | | | | | | | | (Htyr) |
| Ga^{f}/GasNa^{g} | 1 | | —^{e} | | | | | —^{e} | | |
| | 2 | 3.94, m | 73.1, CH | 2-OH, H-3a, H-3b | | 2-OH, H-3a, H-3b, NH (Ala) | 4.11, br s | 71.5, CH | 2-OH, H-3a, H- | 2-OH, H-3a, H-3b, NH (Ala) |
| | | | | | | | | | 3b | |
| | 2-OH | 5.70, d (5.4) | | H-2 | | H-2, H-3a, NH (Ala) | 5.94, br s | | H-2 | H-2, H-3a, H-3b, NH (Ala) |
| | 3a | 3.61, m | 64.4, CH₂ | H-2, H-3b | | H-2, 2-OH, H-3b, H₃-3 (Ala), NH (Ala) | 4.03, d (−10) | 68.9, CH₂ | H-2, H-3b | H-2, 2-OH, H-3b, NH (Ala) |
| | 3b | 3.51, m | | H-2, H-3a | | H-2, 2-OH, H-3a, NH (Ala) | 3.75, m | | H-2, H-3a | H-2, 2-OH, H-3a |

^{a}1-mm HTS cryoprobe.
^{b}Deduced from HSQC and/or HMBC spectra.
^{c}Protons showing HMBC correlations to the indicated carbon.
^{d}Refers to nuclei within the same unit unless indicated otherwise.
^{e}Could not be detected due to lack of HMBC correlation.
^{f}Refers to lyngbyastatin 5 (1).
^{g}Refers to lyngbyastatin 6 (2).
^{h}Proton showed weak TOCSY correlations to H-3 and H₃-4 of the Abu unit. [symbol]
indicates data missing or illegible when filed
FIG. 2. NMR Spectral Data for Lyngbyastatin 7 (3) in DMSO-d₆ (500 MHz)

| Unit | C/H no. | δ_{H} (J in Hz) | δ_{C}, mult. | COSY | HMBC^{a,b} | ROESY |
|---|---|---|---|---|---|---|
| Val | 1 | | 173.9, qC | | | |
| | 2 | 4.72, br | 56.1, CH | H-3, NH | 1 (N—Me- | H-3, H₃-4, H₃-5, NH |
| | | | | | Tyr) | |
| | 3 | 2.09, m | 30.9, CH | H₃-4, H₃-5 | 1, 2, 4, 5 | H-2, H₃-4, H₃-5, NH |
| | 4 | 0.87, d (6.8) | 19.3, CH₃ | H-3 | 2, 3, 5 | H-2, H-3, H₃-5, NH, N—Me (N—Me-Tyr), |
| | | | | | | H₃-4 (Thr) |
| | 5 | 0.75, d (6.8) | 17.5, CH₃ | H-3 | 2, 3, 4 | H-2, H-3, H₃-4, NH, N—Me (N—Me-Tyr) |
| | NH | 7.48, br d (8.5) | | H-2 | | H-2, H-3, H₃-4, H₃-5, N—Me (N—Me-Tyr), |
| | | | | | | H-2 (N—Me-Tyr), 6-OH (Ahp) |
| N—Me-Tyr | 1 | | 169.4, qC | | | |
| | 2 | 4.89, d (11.7) | 60.8, CH | H-3a, H-3b | 1, 3, 4 | H-3a, H-3b, N—Me, H-5/9, H-2 (Phe), |
| | | | | | | H-3b (Phe), H-5/9 (Phe), NH (Val) |
| | 3a | 3.08, d (−13.5) | 32.8, CH₂ | H-2, H-3b | 2, 5/9 | H-2, H-3b, H-5/9, H-2 (Phe) |
| | 3b | 2.70, dd (−13.5, 11.7) | | H-2, H-3a | 1, 2, 5/9 | H-2, H-3a, H-5/9 |
| | 4 | | 127.8, qC | | | |
| | 5/9 | 6.98, d (8.4) | 130.5, CH | H-5/9 | 4, 5, 7, 8 | H-2, H-3a, H-3b, H-6/8, N—Me, H-2 (Phe) |
| | 6/8 | 6.76, d (8.4) | 115.3, CH | H-6/8 | 4, 7, 9 | H-5/9, 7-OH, H-5/9 (Phe) |
| | 7 | | 156.2, qC | | | |
| | 7-OH | 9.38, s | | | 7, 5/9 | H-6/8 |
| | N—Me | 2.75, s | 30.4, CH₃ | | 2, 1 (Phe) | H-2, H-5/9, H₃-4 (Val), H₃-5 (Val), NH (Val) |
| Phe | 1 | | 170.5, qC | | | |
| | 2 | 4.74, dd (11.5, 4.4) | 50.3, CH | H-3a, H-3b | 1, 3, 2 (Ahp) | H-3a, H-3b, H-5/9, H-2 (N—Me-Tyr), |
| | | | | | | H-3a (N—Me-Tyr), H-5/9 (N—Me-Tyr), |
| | | | | | | H-3 (Ahp), H-6 (Ahp) |
| | 3a | 2.87, dd (−13.7, 11.5) | 35.3, CH₂ | H-2, H-3b | 2, 4, 5/9 | H-2, H-3b, H-5/9, H-6 (Ahp), 6-OH (Ahp) |
| | 3b | 1.82, dd (−13.7, 4.4) | | H-2, H-3a | 2, 4, 5/9 | H-2, H-3a, H-6 (Ahp), H-2 (N—Me-Tyr) |
| | 4 | | 136.7, qC | | | |
| | 5/9 | 6.84, d (6.9) | 129.4, CH | H-6/8 | 3, 5/9, 7 | H-2, H-3a, H-6/8, H-2 (N—Me-Tyr), |
| | | | | | | H-6/8 (N—Me-Tyr), H-3 (Ahp) |
| | 6/8 | 7.19, m | 127.5, CH | H-5/9 | 4, 6/8 | H-5/9, H-7 |
| | 7 | 7.15, m | 126.3, CH | H-6/8 | 5/9, 6/8 | H-6/8 |
| Ahp | 2 | | 168.9, qC | | | |
| | 3 | 3.80, ddd (11, 9, 6) | 48.2, CH | H-4a, H-4b, NH | 2, 4 | H-4b, H-5a, NH, H-2 (Phe), H-5/9 (Phe), H-3 |
| | | | | | | (Abu) |
| | 4a | 2.41, m | 21.9, CH₂ | H-4b, H-5a, H-5b, H-3 | | H-4b, 6-OH, NH |
| | 4b | 1.57, m | | H-4a, H-5a, H-3 | | H-3, H-4a |
| | 5a | 1.73, m | 29.3, CH₂ | H-4a, H-4b, H-5b, H-6 | | H-3, H-5b, H-6 |
| | 5b | 1.56, m | | H-5a, H-6, H-4a | | H-5a, H-6, 6-OH |
| | 6 | 5.08, s | 73.8, CH | 6-OH, H-5a, H-5b | | H-5a, H-5b, 6-OH, H-2 (Phe), H-3a (Phe), |
| | | | | | | H-3b (Phe) |
| | 6-OH | 6.10, s | | H-6 | | H-4a, H-5a, H-5b, H-6, NH (Val), H-3a (Phe) |
| | NH | 7.18, d (9) | | | | H-3, H-4a, H-4b, NH (Abu) |
| Abu | 1 | | 162.8, qC | | | |
| | 2 | | 130.0, qC | | | |
| | 3 | 6.52, q (6.9) | 131.8, CH | H₃-4 | 1, 2, 4 | H₃-4, H-3 (Ahp) |
| | 4 | 1.49, d (6.9) | 13.1, CH₃ | H-3 | 2, 3 | H-3, NH, H-2 (Thr) |
| | NH | 9.18, br s | | | | H₃-4, H-2 (Thr), NH (Ahp) |
| Thr | 1 | | 173.0, ^{c} qC | | | |
| | 2 | 4.54, br | 55.7, CH | | | H-3, H₃-4, H₃-4 (Abu), NH (Abu) |
| | 3 | 5.48, br | 71.8, CH | H₃-4 | | H₃-4, H-2 |
| | 4 | 1.22, d (6.5) | 18.1, CH₃ | H-3 | 2, 3 | H-2, H-3, H₃-4, H₃-4 (Val), H-2 (Gln), NH |
| | NH | 7.88, br | | H-2 | | H-2 (Gln) |
| Gln | 1 | | 172.7, qC | | | |
| | 2 | 4.40, ddd (8, 8, 6) | 52.2, CH | | 1, 4 | H-3a, H-3b, H₂-4, H₃-4 (Thr), NH (Thr), |
| | | | | | | H₂-3 (Ha) |
| | 3a | 1.92, m | 26.9, CH₂ | H-3b, H-2, H₂-4 | | H-2, H-3b, H₂-4 |
| | 3b | 1.72, m | | H-3a, H-2, H₂-4 | 1, 2, 4, 5 | H-2, H-3a, H₂-4, 2-NH, H₂-2 (Ha) |
| | 4 | 2.13, m (2H) | 31.5, CH₂ | H-3a, H-3b | 5 | H-2, H-3b, 2-NH, 5-NHa |
| | 5 | | 173.8, qC | | | |
| | 2-NH | 8.08, br s | | H-2 | | H-3b, H₂-4, H₂-2 (Ha) |
| | 5-NHa | 7.23, br s | | | 5 | H₂-4, H₂-2 (Ha) |
| | 5-NHb | 6.73, br s | | | 4 | |
| Ha | 1 | | 172.5, qC | | | |
| | 2 | 2.14, m | 35.1, CH₂ | H₂-3 | 1, 3 | H₂-3, H₂-4/5, H-3b (Gln), 2-NH (Gln), 5-NHa |
| | | | | | | (Gln) |
| | 3 | 1.5, m (2H) | 24.9, CH₂ | H-2a, H-2b, H₂-4, H₂-5 | 2, 4, 5 | H₃-6, H-2a, H-2b, H₂-4/5, 2-NH (Gln), |
| | | | | | | H-2 (Gln), H₂-4 (Gln) |
| | 4 | 1.28, m (2H) | 30.9, CH₂ | H₂-3 | 3 | H-2a, H-2b, H₂-3, H₂-5, H₃-6 |
| | 5 | 1.28, m (2H) | 21.9, CH₂ | H₃-6 | 4, 6 | H-2a, H-2b, H₂-3, H₂-4, H₃-6 |
| | 6 | 0.85, t (7.0) | 13.9, CH₃ | H₂-5 | 4, 5 | H₂-3, H₂-4/5 |

^{a}Protons showing HMBC correlations to the indicated carbon.
^{b}Refers to nuclei within the same unit unless indicated otherwise.
^{c}No HMBC correlation observed.
Carbon assigned to Thr unit based on remaining unassigned signal in the ¹³C NMR (150 MHz).
TABLE 3
NMR data for both conformers of kempopeptin A (5) in DMSO-d₆ (ratio 1:1) at 500 MHz (¹H) and 150 MHz (¹³C)

| | C/H | Trans conformer^{a} | | Cis conformer^{a} | | | |
|---|---|---|---|---|---|---|---|
| Unit | no. | δ_{H} (J in Hz) | δ_{C}, mult. | δ_{H} (J in Hz) | δ_{C}, mult. | HMBC^{b,c} | Key ROESY^{c} |
| Val | 1 | | 172.1, qC | | 172.1, qC | | |
| | 2 | 4.65, dd (9.2, 4.5) | 55.8, CH | 4.64, dd (9.5, 4.5) | 55.8, CH | 1, 3, 4, 5, 1 | |
| | | | | | | (N—Me-Tyr) | |
| | 3 | 2.04, m | 31.8, CH | 2.04, m | 30.8, CH | 2, 4, 5 | |
| | 4 | 0.85, d (6.5) | 19.5, CH₃ | 0.84, d (6.5) | 19.3, CH₃ | 2, 3, 5 | N—Me (N—Me-Tyr) |
| | 5 | 0.71, d (6.5) | 17.2, CH₃ | 0.70, d (6.5) | 17.2, CH₃ | 2, 3, 4 | N—Me (N—Me-Tyr) |
| | NH | 7.43, d (9.2) | | 7.42, d (9.5) | | 1 (N—Me-Tyr) | H-2 (N—Me-Tyr), N—Me (N—Me- |
| | | | | | | | Tyr), 6-OH (Ahp) |
| N—Me-Tyr | 1 | | 169.1, qC | | 169.1, qC | | |
| | 2 | 4.89, dd (10.6, 1.5) | 60.9, CH | 4.89, dd (10.6, 1.5) | 60.9, CH | | H-3a, N—Me, H-2 (Phe), H-5/9 |
| | | | | | | | (Phe), NH (Val) |
| | 3a | 3.10, dd (−13, 10.6) | 32.8, CH₂ | 3.10, dd (−13, 10.6) | 32.8, CH₂ | 4, 5/9 | H-2 |
| | 3b | 2.69, dd (−13, 1.5) | | 2.69, dd (−13, 1.5) | | 4, 5/9 | |
| | 4 | | 127.5, qC | | 127.5, qC | | |
| | 5/9 | 6.99, d (8.5) | 130.4, CH | 6.99, d (8.5) | 130.4, CH | 3, 5/9, 7 | N—Me, H-2 (Phe) |
| | 6/8 | 6.77, d (8.5) | 115.3, CH | 6.77, d (8.5) | 115.3, CH | 4, 6/8, 7 | |
| | 7 | | 156.2, qC | | 156.2, qC | | |
| | 7-OH | 9.35, s | | | | 6/8, 7 | |
| | N—Me | 2.75, s | 30.3, CH₃ | 2.75, s | 30.3, CH₃ | 2, 1 (Phe) | H-2, H-5/9, H₃-4 (Val), H₃-5 |
| | | | | | | | (Val), NH (Val) |
| Phe | 1 | | 170.4, qC | | 170.4, qC | | |
| | 2 | 4.73, dd (11.5, 4.3) | 50.3, CH | 4.73, dd (11.5, 4.3) | 50.3, CH | 1, 2 (Ahp), 6 | H-3b, H-5/9, H-2 (N—Me-Tyr), |
| | | | | | | (Ahp) | H-5/9 (N—Me-Tyr), H-6 (Ahp) |
| | 3a | 2.85, dd (−13.8, 11.5) | 35.3, CH₂ | 2.85, dd (−13.8, 11.5) | 35.3, CH₂ | 2, 4 | |
| | 3b | 1.77, dd (−13.8, 4.3) | | 1.77, dd (−13.8, 4.3) | | 2, 4 | H-2 |
| | 4 | | 136.7, qC | | 136.7, qC | | |
| | 5/9 | 6.82, d (7.0) | 129.4, CH | 6.82, d (7.0) | 129.4, CH | 3, 5/9, 6/8 | H-2, H-2 (N—Me-Tyr) |
| | 6/8 | 7.15, m | 127.7, CH | 7.15, m | 127.7, CH | 4, 7 | |
| | 7 | 7.12, m | 126.2, CH | 7.12, m | 126.2, CH | 5/9, 6/8 | |
| Ahp | 2 | | 168.9, qC | | 168.9, qC | | |
| | 3 | 3.61, m | 48.6, CH | 3.61, m | 48.6, CH | 2 | H-4b, H-5a, NH |
| | 4a | 2.37, m | 21.7, CH₂ | 2.37, m | 21.7, CH₂ | | H-4b, 6-OH, NH |
| | 4b | 1.55, m | | 1.55, m | | | H-3, H-4a |
| | 5a | 1.66, m | 29.0, CH₂ | 1.66, m | 29.3, CH₂ | | H-3, H-5b, H-6, 6-OH |
| | 5b | 1.54, m | | 1.54, m | | | H-5a, H-6, 6-OH |
| | 6 | 5.05, br s | 73.7, CH | 5.05, br s | 73.7, CH | | H-5a, H-5b, 6-OH, H-2 (Phe) |
| | 6-OH | 6.02, br s | | 6.02, br s | | | H-4a, H-5a, H-5b, H-6, NH |
| | | | | | | | (Val) |
| | NH | 7.06, d (9.1) | | 7.06, d (9.1) | | 1 (Leu) | H-3, H-4a, H-2 (Leu) |
| Leu | 1 | | 170.1, qC | | 170.1, qC | | |
| | 2 | 4.19, m | 50.3, CH | 4.19, m | 50.3, CH | | H-3a, H₃-5, NH, NH (Ahp) |
| | 3a | 1.70, m | 40.0, CH₂ | 1.70, m | 40.0, CH₂ | 5, 6 | H-2 |
| | 3b | 1.28, m | | 1.28, m | | | NH |
| | 4 | 1.44, m | 23.3, CH | 1.44, m | 23.3, CH | | NH |
| | 5 | 0.70, d (6.4) | 20.9, CH₃ | 0.70, d (6.4) | 20.9, CH₃ | 3, 4, 6 | H-2 |
| | 6 | 0.83, d (6.4) | 21.5, CH₃ | 0.83, d (6.4) | 21.5, CH₃ | 3, 4, 5 | |
| | NH | 8.40, d (8.7) | | 8.39, d (8.8) | | 1 (Thr-1) | H-2, H-3b, H-4, H-2 (Thr-1), H- |
| | | | | | | | 3 (Thr-1) |
| Thr-1 | 1 | | 169.2, qC | | 169.2, qC | | |
| | 2 | 4.58, dd (9.1, 2.1) | 54.6, CH | 4.60, dd (9.1, 2.0) | 54.6, CH | 1, 1 (Thr-2) | H₃-4, NH (Leu) |
| | 3 | 5.38, br q (6.4) | 72.0, CH | 5.39, br q (6.4) | 72.0, CH | 1 (Val) | NH (Leu) |
| | 4 | 1.18, d (6.4) | 17.7, CH₃ | 1.17, d (6.4) | 17.7, CH₃ | | |
| | NH | 7.62, d (9.1) | | 7.75, d (9.1) | | 1 (Thr-2) | H-2 (Thr-2), H-3 (Thr-2), H₃-4 |
| | | | | | | | (Thr-2) |
| Thr-2 | 1 | | 170.7, qC | | 170.6, qC | | |
| | 2 | 4.30, dd (8.2, 4.1) | 58.1, CH | 4.39, dd (8.4, 4.2) | 58.0, CH | 1 | NH (Thr-1) |
| | 3 | 4.03, m | 66.5, CH | 4.03, m | 66.5, CH | | NH (Thr-1) |
| | 4 | 1.02, t (6.7) | 19.2, CH₃ | 1.04, t (6.8) | 19.3, CH₃ | | NH (Thr-1) |
| | OH | 4.86, d (5.1) | | 4.96, d (5.4) | | | |
| | NH | 7.91, d (8.2) | | 8.12, d (8.4) | | 1 (Pro) | H-2 (Pro) |
| Pro | 1 | | 172.3, qC | | 172.1, qC | | |
| | 2 | 4.44, dd (8.4, 2.8) | 58.9, CH | 4.52, dd (8.6, 2.9) | 60.1, CH | | NH (Thr-2) |
| | 3a | 2.05, m | 29.3, CH₂ | 2.23, m | 29.5, CH₂ | | |
| | 3b | 1.90, m | | 1.93, m | | | |
| | 4 | 1.89, m | 24.3, CH₂ | 1.76, m | 24.1, CH₂ | | |
| | 5a | 3.51, m | 47.6, CH₂ | 3.40, m | 46.3, CH₂ | | |
| | 5b | 3.47, m | | 3.38, m | | | |
| Ac | 1 | | 168.7, qC | | 168.5, qC | | |
| | 2 | 1.95, s | 22.0, CH₃ | 1.83, s | 22.2, CH₃ | 1 | H-2^{d} (Pro)^{cis} or H₂-5^{e} (Pro)^{trans} |

^{a}Refers to restricted rotation around the N-acyl-prolyl amide bond.
^{b}Protons showing HMBC correlations to the indicated carbon.
^{c}Refers to nuclei within the same unit unless indicated otherwise.
^{d}Refers to cis isomer.
^{e}Refers to trans isomer.
TABLE 4
NMR data for kempopeptin B (6) in DMSO-d₆ at 600 MHz (¹H) and 150 MHz (¹³C)

| | C/H | | | | | |
|---|---|---|---|---|---|---|
| Unit | no. | δ_{H} (J in Hz) | δ_{C}, mult. | COSY^{a} | HMBC^{b,c} | Key ROESY^{a,c} |
| Val-1 | 1 | | 172.3, qC | | | |
| | 2 | 4.63, dd (9.4, 5.5) | 54.8, CH | H-3, NH | 1, 1 | |
| | | | | | (N,O-diMe-Br- | |
| | | | | | Tyr) | |
| | 3 | 2.04, m | 30.5, CH | H₃-4, H₃-5 | 1, 2, 4, 5 | |
| | 4 | 0.85, d (6.8) | 19.3, CH₃ | H-3 | 2, 3, 5 | N—Me (N,O-diMe-Br-Tyr) |
| | 5 | 0.73, d (6.8) | 17.6, CH₃ | H-3 | 2, 3, 4 | N—Me (N,O-diMe-Br-Tyr) |
| | NH | 7.68, d (9.4) | | H-2 | 1 (N,O-diMe- | H-2 (N,O-diMe-Br-Tyr), N—Me |
| | | | | | Br-Tyr) | (N,O-diMe-Br-Tyr), 6-OH (Ahp) |
| N,O-diMe-Br-Tyr | 1 | | 169.4, qC | | | |
| | 2 | 5.03, dd (11.3, | 60.6, CH | H-3a, H-3b | 3 | H-3a, H-5, H-9, N—Me, H-2 (Ile), |
| | | 2.6) | | | | NH (Val-1) |
| | 3a | 3.20, dd (−12.0, | 32.9, CH₂ | H-2, H-3b | | H-2, H-5 |
| | | 2.6) | | | | |
| | 3b | 2.78, dd (−12.0, | | H-2, H-3a | | |
| | | 11.3) | | | | |
| | 4 | | 131.3, qC | | | |
| | 5 | 7.39, d (1.8) | 133.5, CH | H-9 | 3, 6, 7, 9 | H-2, H-3a, N—Me, H-2 (Ile) |
| | 6 | | 111.0, CH | | | |
| | 7 | | 154.6, qC | | | |
| | 8 | 7.01, d (8.4) | 113.0, CH | H-9 | 4, 6, 7 | |
| | 9 | 7.17, dd (8.4, 1.8) | 130.2, CH | H-5, H-8 | 3, 5, 7 | H-2 |
| | O—Me | 3.74, s | 56.1, CH₃ | | 7 | |
| | N—Me | 2.72, s | 30.2, CH₃ | | 2, 1 (Ile) | H-2, H-5, H₃-4 (Val-1), H₃-5 |
| | | | | | | (Val-1), NH (Val-1) |
| Ile | 1 | | 169.7, qC | | | |
| | 2 | 4.35, br d (10.7) | 54.2, CH | H-3 | 1, 3, 6 (Ahp) | H-3, H-5, H₃-6, H-2 (N—Me—Br- |
| | | | | | | Tyr), H-5 (N,O-diMe-Br-Tyr) |
| | 3 | 1.79, m | 32.8, CH | H-2, H-4a, H-4b, H₃-6 | | H-2 |
| | 4a | 1.0, m | 23.7, CH₂ | H-3, H-4b, H₃-5 | | |
| | 4b | 0.629, m | | H-3, H-4a, H₃-5 | | |
| | 5 | 0.630, br t (6.9) | 10.3, CH₃ | H-4a, H-4b | 3, 4 | H-2 |
| | 6 | −0.15, d (6.4) | 13.8, CH₃ | H-3 | 2, 3, 4 | H-2 |
| Ahp | 2 | | 170.4,^{d} qC | | | |
| | 3 | 4.42, m | 48.8, CH | H-4a, H-4b, NH | | H-4b, H-5, NH |
| | 4a | 2.55, m | 21.7, CH₂ | H-3, H-4b, H-5 | | H-4b, 6-OH, NH |
| | 4b | 1.71, m | | H-3, H-4a, H-5 | | H-3, H-4a |
| | 5 | 1.73, m (2H) | 29.7, CH₂ | H-4a, H-4b, H-6 | | H-3, H-6 |
| | 6 | 4.92, d (2.9) | 74.1, CH | H-5, 6-OH | | H-5, 6-OH |
| | 6-OH | 6.15, d (2.9) | | H-6 | | H-4a, H-6, NH (Val-1) |
| | NH | 7.34, d (9.3) | | H-3 | | H-3, H-4a, H-2 (Lys) |
| Lys | 1 | | 169.3, ^{d} qC | | | |
| | 2 | 4.26, br | 52.1, qC | H-3a, H-3b, NH | | H-3a, H-4, NH, NH (Ahp) |
| | 3a | 2.00, m | 29.0, CH₂ | H-2, H-3b, H₂-4 | | H-2 |
| | 3b | 1.41, m | | H-2, H-3a, H₂-4 | | NH |
| | 4 | 1.23, m (2H) | 22.1, CH₂ | H-3a, H-3b, H₂-5 | | H-2 |
| | 5 | 1.47, m (2H) | 26.3, CH₂ | H₂-4, H₂-6 | | |
| | 6 | 2.71, m (2H) | 38.6, CH₂ | H₂-5 | | |
| | NH | 8.44, d (8.4) | | H-2 | 1 (Thr) | H-2, H-3b, H-2 (Thr), H-3 (Thr), |
| | | | | | | NH (Thr) |
| | NH₂ | 7.60, br s (2H) | | | | |
| Thr | 1 | | 169.3, qC | | | |
| | 2 | 4.59, dd (10.2, | 56.3, CH | H-3, NH | 1 (Val-2) | H-3, H-4, NH (Lys), NH (Val-2) |
| | | 6.5) | | | | |
| | 3 | 5.49, br q (6.5) | 71.7, CH | H-2, H₃-4 | 4, 1 (Val-1) | H-2, NH (Lys) |
| | 4 | 1.20, d (6.5) | 17.7, CH₃ | H-3 | 2, 3 | H-2 |
| | NH | 7.80, d (10.2) | | H-2 | 1 | NH (Lys) |
| Val-2 | 1 | | 172.4, qC | | | |
| | 2 | 4.31, dd (9, 7.1) | 57.6, CH | H-3, NH | 1 | H-3, NH, H₃-4 (Ba) |
| | 3 | 2.01, m | 30.0, CH | H₃-4, H₃-5 | | H-2 |
| | 4 | 0.83, d (7.6) | 19.3, CH₃ | H-3 | 2 | |
| | 5 | 0.82, d (7.6) | 18.1, CH₃ | H-3 | 2 | |
| | NH | 7.82, d (9) | | H-2 | 1 (Ba) | H-2, H-2 (Thr), H₂-2 (Ba) |
| Ba | 1 | | 172.5, qC | | | |
| | 2 | 2.16, m (2H) | 37.1, CH₂ | H₂-3 | 1, 3, 4 | H₃-4, NH (Val-2) |
| | 3 | 1.49, m (2H) | 18.9, CH₂ | H₂-2, H₃-4 | 1, 2, 4 | |
| | 4 | 0.84, t (7.5) | 13.6, CH₃ | H₂-3 | 2, 3 | H₂-2, H-2 (Val-2) |

^{a}Recorded at 500 MHz.
^{b}Protons showing HMBC correlations to the indicated carbon (600 MHz).
^{c}Refers to nuclei within the same unit unless indicated otherwise.
^{d}Interchangeable.
No HMBC correlations observed. Carbons assigned based on remaining unassigned signals in the ¹³C NMR spectrum.
TABLE 5
Protease inhibitory activity (IC₅₀) from metabolites
isolated from the Lyngbya sp. from Kemp Channel

| | Elastase | | Chymotrypsin | | Trypsin | |
|---|---|---|---|---|---|---|
| Kempopeptin A | 320 ± 70 | nM | 2,600 ± 100 | nM | >67,000 | nM |
| (1) | | | | | | |
| Kempopeptin B | >67,000 | nM | >67,000 | nM | 8,400 ± 200 | nM |
| (2) | | | | | | |
| Lyngbyastatin 7^{a} | 8.3 ± 5.4 | nM | 2,500 ± 200 | nM | >30,000 | nM |
| Somamide B^{a} | 9.5 ± 5.2 | nM | 4,200 ± 500 | nM | >30,000 | nM |

^{a}Taken from Taori, K.; Matthew, S.; Rocca, J. R.; Paul, V. J.; Luesch, H. J. Nat. Prod. 2007, 70, 1593-1600.
